SEQUENCE LISTING (2 sequences)

SEQ ID NO: 1 - DNA, 1769 bp, Homo sapiens
  CDS (88)..(933)
cctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagaaaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtgcaaaaattagctgggtgtgttggcacatgcctgtagtcccagctacttgagaggctgaggcaggagaatcgtttgaacccgggaggcagaggttgcagtgtggtgagatcatgccactacactccagcctggcgacagagcgagacttggtttcaaaaaaaaaaaaaaaaaaaaacttcagtaagtacgtgttatttttttcaataaaattctattacagtatgtcaaaaaaaaaaaaaaaaaa

SEQ ID NO: 2 - protein, 281 aa, Homo sapiens [sequence omitted]

(19) United States
(12) Patent Application Publication
(10) Pub. No.: US 2004/0213764 A1
(43) Pub. Date: Oct. 28, 2004

(54) Adenovirus replication-competent vectors expressing trail

(76) Inventors: William Wold, Chesterfield, MO (US); Ann Tollefson, St. Louis, MO (US); Baoling Ying, Ballwin, MO (US)

Correspondence Address: FULBRIGHT & JAWORSKI L.L.P., 600 CONGRESS AVE., SUITE 2400, AUSTIN, TX, 78701, US

(21) Appl. No.: 10/810,063

(22) Filed: Mar. 26, 2004

Related U.S. Application Data
(60) Provisional application No. 60/458,493, filed on Mar. 28, 2003.

(51) Int. Cl. A61K 48/00; C12N 15/861

(52) U.S. Cl. 424/93.2; 435/456

(57) ABSTRACT

The present invention provides replication-competent Adenoviral vectors that express TRAIL and ADP. In a particular aspect, TRAIL and/or ADP are highly expressed using the Adenovirus major late promoter or other promoter. Methods of treating hyperproliferative disorders using such vectors, alone or in combination with secondary therapies, also are described.

The present invention claims the benefit of the filing date of U.S. Provisional Patent Application Ser. No. 60/458,493 filed Mar. 28, 2003. The entire text of the above-referenced disclosure is specifically incorporated herein by reference.

This invention was made with government support under Grant Nos. USPHS CA58538, NIH 1R41 CA81829 and NIH 2R42 CA81829 awarded by the NIH. The government has certain rights in the invention.

### BACKGROUND OF THE INVENTION

1. Field of the Invention

The present invention relates generally to the fields of oncology, molecular biology and gene therapy. More particularly, it concerns replication-competent adenoviruses that express TRAIL, and methods of use in anti-proliferative therapies.

2. Description of Related Art

One of the new experimental approaches for treatment of cancer involves exploitation of the cytolytic capacity of adenoviruses. Adenoviruses induce cell death by cytolysis as part of their normal life cycle (Webb and Smith, 1970). Between 1950 and 1975 a number of replicating viruses, including Adenoviruses, were studied in cancer patients (Smith et al., 1956). However, as a cancer treatment, virotherapy was abandoned because only a few clinical responses were reported, their effects were unpredictable, and it was supplanted by more active chemotherapeutic drugs. Adenovirus cancer therapy is now being re-evaluated in the light of recent findings that genetically modified oncolytic (replication-competent) viruses can be rendered cancer-selective (reviewed in Alemany et al., 2000).

The first cancer-selective, replication-competent oncolytic Adenovirus of its generation was ONYX-015 (Bischoff et al., 1996) or CI-1042 (Pfizer Corp., New York, N.Y.). ONYX-015 does not synthesize EIB-55K, a protein that binds and inactivates the tumor suppressor protein p53 and represses p53-responsive promoters (Martin and Berk, 1999), thereby inhibiting p53-induced apoptosis and enabling cells to enter S-phase. It was hypothesized that ONYX-015 would be unable to inactivate p53 in normal cells and would, thus, be unable to replicate efficiently. However, it is now clear that replication of ONYX-015 can be p53-independent (Goodrum and Omelles, 1998; Harada and Berk, 1999). When used alone, objective tumor responses were seen in less than 15% of patients (Khuri et al., 2000).

Oncolytic therapy for cancer will require as much destruction of tumor cells in the body as possible, and therefore efforts have been made to improve these vectors. A polylysine tail has been added to the fiber gene of ONYX-015, resulting in more efficient infection of gliomas (Shinoura et al., 1999). Other attempts aimed at directly increasing cytotoxicity of Adenovirus treatment by combination with chemotherapeutic agents (Kim et al., 1998), radiotherapy (Rogulski et al., 2000), and heat shock (Haviv et al., 2001) treatments are currently under evaluation in clinical trials.

An alternative approach is to develop virus vectors that overexpress endogenous or exogenous cytotoxic genes. The inventors previously constructed tumor-selective, replication-competent Adenovirus vectors that markedly overexpress the Adenovirus ADP. ADP is required for Adenovirus-infected cells to lyse efficiently and for Adenovirus to spread efficiently from cell-to-cell (Tollefson et al., 1996a). Overexpression of ADP by Adenovirus result in a greater cytolytic activity and highly increased cell-to-cell spread (Doronin et al., 2000; 2001). Other Adenovirus vectors, both replication-defective (reviewed in Wildner, 1999) and replication-competent (reviewed in Ring, 2002), have been engineered to express exogenous suicide genes that convert a prodrug into a cytotoxic agent. These strategies also utilize a significant bystander effect of the active drug (Wilder and Norris, 2000; Freytag et al., 1998; Djeha et al., 2001).

A specific type of exogenous cytotoxic gene is a cytokine (Hawkins and Hermiston, 2001). Replication defective Adenoviruses expressing Fas ligand have been shown to be effective against tumors in vivo (Arai et al., 1997). However, Fas-ligand is cytotoxic to many tissues, and so application of this approach in human therapy is unlikely. On the other hand, Tumor Necrosis Factor (TNF)-Related Apoptosis-Inducing Ligand (TRAIL) selectively kills tumor cells. Recently, replication-defective Adenovirus vectors expressing TRAIL have been described (Griffith and Broghammer, 2001; Griffith et al., 2000; Kagawa et al., 2001). These viruses caused significant growth retarding effect in mouse xenotransplanted tumors. Nonetheless, improvements in TRAIL delivery by therapeutic vectors is desired.

### SUMMARY OF THE INVENTION

Thus, in accordance with the present invention, there is provided a replication-competent adenovirus vector comprising a tumor necrosis factor-related apoptosis-inducing ligand (TRAIL) coding region and an ADP coding region. The TRAIL coding region and ADP coding region may be positioned under the control of adenovirus major late promoter (MLP), and may be positioned in the E3 region of the vector. The TRAIL coding region may be positioned upstream or downstream of the ADP coding region. The TRAIL coding region may be positioned under the control of adenovirus major late promoter (MLP), and the ADP coding region may be positioned under the control of another promoter, or vice versa. The vector may lack one or more of coding regions for the 6.7K, gp19K, RIDα, RIDβ or 14.7K proteins, including all of these coding regions. The vector may further comprise at least a first mutation in the E1A region, the mutation impairing binding of E1A to p300 and/or pRB. The vector may be oncolytic. Also provided are an adenoviral virion comprising a replication-competent adenoviral vector as described above, and a host cell comprising the replication-competent adenoviral vector as described above.

In another embodiment, there is provided a method of inhibiting a hyperproliferative cell comprising contacting the cell with a second cell infected with a replication-competent adenovirus vector comprising a tumor necrosis factor-related apoptosis-inducing ligand (TRAIL) coding region and an ADP coding region. Inhibiting may comprise inhibiting cell division, inhibiting cell growth, inducing cell cycle arrest, inducing apoptosis or lysing. The hyperproliferative cell may be a cancer cell, and the second cell may be a cancer cell. Examples of a cancer cell as in the present invention may be a lung cancer cell, a prostate cancer cell, a colon cancer cell, an ovarian cancer cell, a testicular cancer cell, a brain cancer cell, a stomach cancer cell, a uterine cancer cell, a breast cancer cell, an esophageal cancer cell, a head & neck cancer cell, a pancreatic cancer cell, a liver cancer cell, a kidney cancer cell, a skin cancer cell or a blood cancer cell.

In yet another embodiment, there is provided a method of treating a subject with a hyperproliferative cell disorder comprising administering to the subject a replication-competent adenovirus vector comprising a tumor necrosis factor-related apoptosis-inducing ligand (TRAIL) coding region and an ADP coding region. The hyperproliferative disorder may be a cancer, such as lung cancer, prostate cancer, colon cell, ovarian cancer, testicular cancer, brain cancer, stomach cancer, uterine cancer, breast cancer, esophageal cancer, head & neck cancer, pancreatic cancer, liver cancer, kidney cancer, skin cancer or blood cancer. The cancer may be a drug or multi-drug resistant cancer. The subject may be a human. Treating may comprise reducing tumor size, reducing tumor growth, inducing remission, inducing tumor necrosis, or prolonging patient survival.

The method may further comprise administering to the subject a second therapy, such as chemotherapy, radiotherapy, immunotherapy, hormonal therapy, gene therapy or surgery. The second therapy may be provided prior to the replication-competent adenovirus vector, after the replication-competent adenovirus vector, or at the same time as the replication-competent adenovirus vector. The replication-competent adenovirus vector may be administered more than once. The replication-competent adenovirus vector may be administered intratumorally, local to the tumor, regional to the tumor or systemically, intravenously, intraarterially, intramuscularly, intralymphatically, intraperitoneally or subcutaneously. The TRAIL coding region and ADP coding region may be positioned under the control of adenovirus major late promoter (MLP), and may be positioned in the E3 region of the vector. The TRAIL coding region may be positioned upstream or downstream of the ADP coding region. The TRAIL coding region may be positioned under the control of adenovirus major late promoter (MLP), and the ADP coding region may be positioned under the control of another promoter, or vice versa. The vector may lack one or more of coding regions for the 6.7K, gp19K, RIDα, RIDβ or 14.7K proteins, including all of these coding regions. The vector may further comprise at least a first mutation in the E1A region, the mutation impairing binding of E1A to p300 and/or pRB. The vector may be oncolytic.

In further particular embodiments, the present invention provides a method of rendering an inoperable tumor operable comprising administering to a subject a replication-competent adenovirus vector comprising a tumor necrosis factor-related apoptosis-inducing ligand (TRAIL) coding region and an ADP coding region.

In yet another particular embodiment, the present invention provides a method of treating metastatic cancer in a subject comprising administering to subject a replication-competent adenovirus vector comprising a tumor necrosis factor-related apoptosis-inducing ligand (TRAIL) coding region and an ADP coding region.

In still yet another particular embodiment of the invention, there is provided a method of preventing cancer in a subject at risk thereof comprising administering to the subject a replication-competent adenovirus vector comprising a tumor necrosis factor-related apoptosis-inducing ligand (TRAIL) coding region and an ADP coding region; and

In a still further particular embodiment, the present invention provides a method of treating recurrent cancer in a subject comprising administering to the subject a replication-competent adenovirus vector comprising a tumor necrosis factor-related apoptosis-inducing ligand (TRAIL) coding region and an ADP coding region.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Schematic representation of the TRAIL-expressing Adenovirus vectors, and description of the cell line that was used to make the vectors. FIG. 1A. Schematic of the vectors. The vectors are named VRX-013, VRX-014, VRX-015, and VRX-016. The long horizontal bar depicts the double stranded DNA genome of approximately 36,000 base pairs. E1, E2, E3, and E4 refer to transcription units. The white area in which E3 is placed indicates that most of the E3 transcription unit is deleted. The arrow labeled E1A depicts the E1A mRNA from which the E1A proteins are translated. The two “x” marks in the E1A arrows for VRX-013 and VRX-014 depict the two small deletions in the E1A gene. The arrows labeled TRAIL and ADP depict the mRNAs for the TRAIL and ADP proteins. In VRX-013 and VRX-015, the open reading frame (ORF) for TRAIL is located in the genome to the left of the ORF for ADP. In VRX-014 and VRX-016, the ADP ORF is on the left and the TRAIL ORF is on the right. These arrows are connected to three boxes on the left; these boxes depict the tripartite leader which is found at the 5′ termini of all mRNAs in the major late transcription unit. The mRNAs for TRAIL and ADP are expressed from the major late promoter (located at approximate map position 16) by alternative splicing and alternative selection of polyadenylation sites. The tripartite leader at the 5′ termini of these mRNAs facilitates their translation. FIG. 1B. The cell line named 293CrmAE3 expresses the RIDβ and CrmA proteins as determined by western blot. 293CrmA cells are 293 cells that were stably transformed with expression cassettes that express the poxvirus CrmA and the adenovirus E3 RIDα and RIDβ proteins.

FIGS. 2A-2C. TRAIL is expressed and secreted at large quantities in cells infected with the VRX-013 virus. (FIGS. 2A-B) Western blots. A549 cells were infected with 50 PFU of KD3 or VRX-013 per cell. At 24 h and 34 h p.i., the supernatants were collected and cells were lysed. Lysates containing 10 μg of lysate proteins (FIG. 2A) and 6 μl of supernatants (FIG. 2B) were run on a 15% SDS-PAGE gel and western blotted for TRAIL content. Recombinant hTRAIL was used as a control. (FIG. 2C) Immunofluorescence. A549 cells were infected with KD3 or VRX-013 at 10 PFU/cell. At 7 h p.i. cells were treated with ara-C (20 μg/ml) or vehicle, and at 23 h p.i. the cells were immunostained for TRAIL.

FIG. 3. Expression of ADP by VRX-013 and KD3. A549 cells were infected with 50 PFU/cell of VRX-013 or KD3. At 24 h and 34 h p.i., proteins were extracted and analyzed for the ADP and E1A proteins by western blot using antisera specific to ADP and E1A, respectively.

FIGS. 4A-4C. Supernatants from VRX-013-infected A549 or 293 cells have cytotoxic activity, presumably due to the function of TRAIL. (FIG. 4A) Titration of TRAIL expression. A549 and 293 cells were infected with 50 PFU/cell of KD3 or VRX-013. At 24 h supernatants were removed and used to treat A549 cells; cell viability was determined using the MTT assay. Supernatants from KD3-infected cells did not induce cytotoxicity (data not shown). (FIG. 4B) The kinetics of cell death induced by supernatants from VRX-013-infected A549 or 293 cells. A549 cells were treated with a 3.2-fold dilution of A549 or 293 supernatants (see Panel A). Viability at different periods post-treatment, as shown in the graph, was determined using the MTT assay. (FIG. 4C) VRX-013 causes more cell death than KD3 or dl309 in most cancer cell lines. A variety of cancer cell lines were infected with KD3, VRX-013 or dl309 at 10 PFU/cell. dl309 is an Ad5 mutant with a deletion in the E3 region that removes the gene for the RIDα, RIDβ, and 14.7K proteins. dl309 expresses ADP at levels similar to Ad5 (Doronin et al., 2003). Viability relative to mock-infection was determined at 5 d p.i. (SW1116, LS 513, SW480, HepG2), 7 d p.i. (LS 174T), or 8 d p.i. (LNCaP) using the trypan blue exclusion assay for cell permeability.

FIG. 5. VRX-013 induces more cell death in five cancer cell lines at 7 days p.i. than do the viruses that do not express TRAIL. Cells were seeded in 48-well plates and were mock-infected or infected with 10-fold serial dilutions of the indicated viruses, ranging from 10¹ to 10⁴ PFU/cell. Monolayers were fixed and stained with crystal violet at 7 days p.i.

FIG. 6. VRX-013 is much more effective than KD3 in inducing cytopathic effect in Hep3B liver cancer cells at 2 days p.i. Hep3B cells were mock-infected or infected with 10, 10⁰, 10⁻¹, and 10⁻² PFU/cell of VRX-013 or KD3. The cells were photographed under phase contrast at 2 days p.i.

FIG. 7. VRX-013 spreads from cell-to-cell, apparently because TRAIL does not induce apoptosis in infected cells at low MOI; TRAIL, however, induces apoptosis in surrounding cells. A549 cells were infected with 10⁻² PFU/cell of VRX-013 or KD3. At 4 days p.i., cells were fixed in methanol containing DAPI (to stain the DNA in the nucleus), then immunostained for the Adenovirus E2-coded DNA binding protein (DBP). The same field of DAPI- and DBP-stained cells is shown for KD3 (top two panels), and similarly for VRX-013 (bottom two panels). In this figure, the term KD3/TRAIL refers to VRX-013. With KD3 and to a lesser extent with VRX-013, many of the cells are infected (i.e., they express DBP in the nucleus); this suggests that the vectors have spread from a putative single originally infected cell. With VRX-013, the arrows indicate infected cells where the nuclei are not apoptotic. Nearly all the surrounding nuclei are apoptotic.

FIG. 8. VRX-013 infection induces apoptosis in neighboring cells, but not in the originally infected cell. KB, HepG2, or SW1116 cells were infected at low MOI with VRX-013. At 2 days p.i. the cells were fixed and stained with DAPI and immunostained for DBP. Many of the cells are infected as indicated by the immunostaining for DBP (left three panels). As indicated by the DAPI staining (right three panels), many of the uninfected cells have apoptotic nuclei.

FIG. 9. VRX-013 and KD3 are equally efficacious in reducing the growth of Hep3B tumors in nude mice. Subcutaneous Hep3B xenografts were established in nude mice. Three weeks later established tumors of average size of about 300 mm³ were injected intratumorally with 5×10⁹ PFU/injection of KD3, KD3/TRAIL, or vehicle. Injections were repeated 5 times at 2 day intervals (total dose 2.5×10¹⁰ PFU/tumor). Tumor sizes were taken by digital calipers on days post-injection as indicated.

FIG. 10. VRX-014 and VRX-016 express TRAIL as indicated by immunofluorescence staining for TRAIL. A549 cells were infected at 20 PFU/cell with VRX-007, VRX-014, or VRX-016. At 48 h p.i. cells were fixed and immunostained with a rabbit polyclonal antibody specific for human TRAIL. For VRX-014 and VRX-016, TRAIL expression is apparent in Golgi, vesicles, and some plasma membranes.

FIG. 11. Expression of ADP in virus infected DLD-1 cells. DLD-1 cells were mock-infected or infected with the indicated viruses at a MOI of 10 PFU/cell. At 24 h and 48 h p.i., proteins were extracted and ADP was detected by immunoblot. The upper ADP band is the glycosylated form of ADP and the lower band is a proteolytic cleavage product. The bottom panel shows expression of TRAIL.

FIG. 12. Vector spread assay. Monolayers of DLD-1 were infected with serial dilutions (10 PFU/cell to 10⁻⁴ PFU/cell) of the indicated virus vectors in multi-well plates. At 4 days p.i. (top panel) and 8 days p.i. (bottom panel), cells remaining on the plates were fixed and stained with crystal violet.

FIG. 13. VRX-014 and VRX-016 spread from the originally-infected cells as indicated by immunofluorescence staining for the adenovirus E1A protein. DLD-1 and Hep3B tumor cells were infected with VRX-014 and VRX-016 at low multiplicities of infection. At 3.5 days p.i. cells were fixed and immunostained for the adenovirus E1A protein. Intense focal staining indicates viral replication and infection of neighboring cells by progeny virus. For the panel shown, initial infection by VRX-014 was at 2.2×10⁻⁴ PFU/cell (DLD-1) or 6.6×10⁻⁴ PFU/cell (Hep3B). Initial infection by VRX-016 was at 2.4×10⁻³ PFU/cell (DLD-1) or 7.2×10⁻⁴ PFU/cell (Hep3B).

FIG. 14. VRX-014 and VRX-016 express TRAIL which induces apoptosis in Hep3B cells neighboring the infected cells. DLD-1 or Hep3B tumor cells were infected with VRX-014 or VRX-016 at low MOI. At 3 days p.i, small clusters of TRAIL and E1A positive cells are seen. DAPI staining of cells indicates the presence of many apoptotic nuclei in the vicinity of TRAIL-expressing cells (uninfected monolayers are shown for comparison; labeled as “mock”).

FIG. 15. VRX-014 and VRX-016 express TRAIL, which induces apoptosis in DLD-1 cells that neighbor the infected cells. See the legend to FIG. 14 for details.

FIG. 16. VRX-015 expresses TRAIL and induces apoptosis. A549 cells were infected with VRX-015. At 23 h p.i. cells were fixed and immunostained for TRAIL and for the adenovirus protein E1A, as a control for infection. The nuclear DNA was stained with DAPI during fixation. The TRAIL panels indicate that TRAIL is made abundantly in the infected cells. TRAIL is localized in Golgi, vesicles, and the plasma membrane. The DAPI fields are in a higher plane to show the presence of apoptotic nuclei next to the TRAIL-positive cells. Note also that the nuclei of TRAIL expressing cells are not apoptotic.

FIG. 17. Tumor suppression in nude mice.

### DESCRIPTION OF ILLUSTRATIVE EMBODIMENTS

Cancer is a leading cause of death in the United States and elsewhere. Depending on the type of cancer, it is typically treated with surgery, chemotherapy, and/or radiation. These treatments often fail: surgery may not remove all the cancer; some cancers are resistant to chemotherapy and radiation therapy; and chemotherapy-resistant tumors frequently develop. New therapies are necessary, to be used alone or in combination with classical techniques.

One potential therapy under active investigation is treating tumors with recombinant viral vectors expressing anti-cancer therapeutic proteins. Adenovirus-based vectors contain several characteristics that make them conceptually appealing for use in treating cancer, as well as for therapy of genetic disorders: Adenoviruses can easily be grown in culture to high titer stocks that are stable; they have a broad host range, replicating in most human cancer cell types; and their genome can be manipulated by site-directed mutation and insertion of foreign genes expressed from foreign promoters.

Despite these generally positive attributes, it is recognized that replication-defective Adenoviral vectors have several characteristics that make them suboptimal for use in therapy. For example, production of replication-defective vectors requires that they be grown on a complementing cell line that provides the E1A proteins in trans. Such cell lines are fastidious, and generation of virus stocks is time-consuming and expensive. In addition, although many foreign proteins have been expressed from such vectors, the level of expression is low compared to Adenovirus late proteins.

To address these problems, several groups have proposed using replication-competent Adenoviral vectors for therapeutic use. Replication-competent vectors retain Adenovirus genes essential for replication and thus do not require complementing cell lines to replicate. Replication-competent Adenovirus vectors lyse cells as a natural part of the life cycle of the vector. Another advantage of replication-competent Adenovirus vectors occurs when the vector is engineered to encode and express a foreign protein. Such vectors would be expected to greatly amplify synthesis of an foreign protein in vivo as the vector replicates. However, in order to prevent such vectors from damaging normal tissues and causing disseminated viremia, it is important that they have some feature that limits their replication to cancer cells. This in turn can limit their effectiveness.

### I. THE PRESENT INVENTION

The inventors now provide the construction and characterization of a replication-competent Adenovirus vectors that have an apoptosis-inducing capability due to expression and secretion of TRAIL and the expression of ADP, as well as their use in mono- and combo-therapies. In addition, various other engineering steps may be included, such as a mutation in the ElA gene that prevents E1A binding to p300 and Rb, and a deletion in the E3 region that removes one, two, three, four or all of the genes encoding 6.7K, gp19K, RIDα, RIDβ, and 14.7K.

Another important feature is achieving high level expression of TRAIL and/or ADP, which may be accomplished by placing these open reading frames under control of the Adenovirus major late transcription promoter. This transcription unit is highly active late in infection. When ADP was placed in this position, overexpression of ADP was achieved in a virus designated as KD3 (Doronin et al., 2000), and the same is seen with TRAIL here. However, those of skill in the art will be well aware of how to achieve high level expression of one or both genes in the same construct. Thus, the viruses as now disclosed should be superior to prior replication-defective vectors expressing TRAIL.

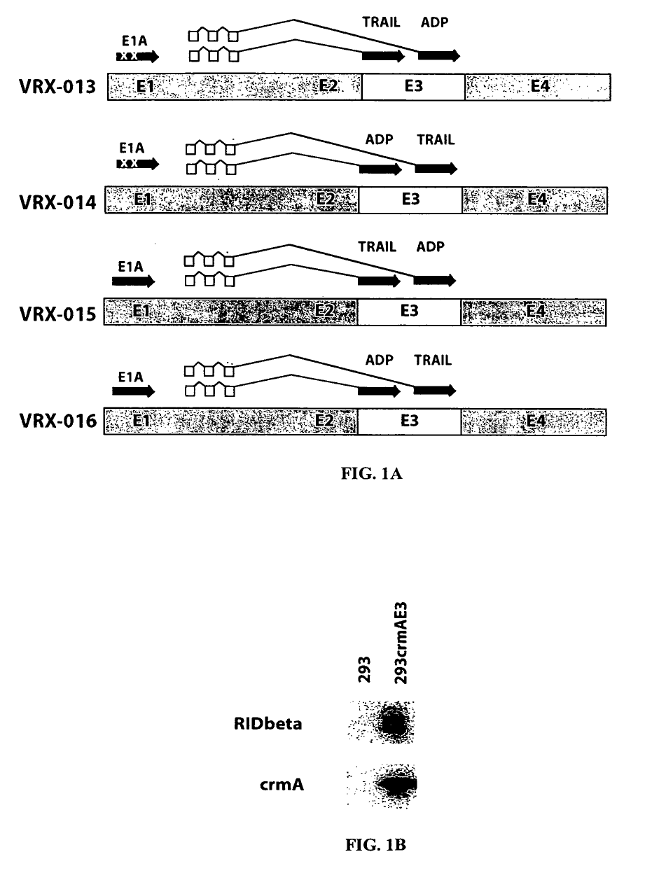

Various permutations of the elements discussed above are provided herein (FIG. 1A). VRX-013 has a mutated E1A gene, E3-deletion, and TRAIL and ADP inserted into the E3 region (in that order). VRX-015 is exactly like VRX-013 except it has a wild-type E1A gene. VRX-015 is expected to express high levels of TRAIL at late stages of infection, as is the case with VRX-013, but to replicate somewhat more efficiently in cancer cell lines than VRX-013 because the E1A proteins are completely wild-type. VRX-015 may be useful in treating cancers that require high levels of TRAIL, and somewhat more replication than VRX-013.

In two additional vectors named VRX-014 and VRX-016, the cDNA for TRAIL is inserted into the E3 region at a site downstream of the gene for ADP. VRX-014 has in the same ElA background as VRX-013 and KD3, i.e., it has the mutation in the E1A region such that the E1A proteins do not bind p300 and pRB. VRX-016 has a wild-type E1A gene, as is the case with VRX-015. The key design feature of VRX-014 and VRX-016 is that ADP is synthesized at high levels similar to that in KD3, and that TRAIL is synthesized in somewhat lower levels. Because ADP is overexpressed in VRX-014 and VRX-016, these viruses spread from cell-to-cell efficiently, in fact at rates that are comparable to that of KD3. VRX-014 and VRX-016 may be useful in cancer treatment situations in which spread of the vector throughout the tumor is desired, but with somewhat lower levels of TRAIL synthesis than with VRX-013 or VRX-015.

These embodiments, and various other aspects of the invention, are described in the following pages.

### II. ADENOVIRUS AND ENGINEERED ADENOVIRAL VECTORS

The Adenovirion consists of a DNA-protein core within a protein capsid (reviewed by Stewart et al.). Virions bind to a specific cellular receptor, are endocytosed, and the genome is extruded from endosomes and transported to the nucleus. The genome is a linear duplex DNA of about 36 kbp, encoding about 36 genes.

In the nucleus, the “immediate early” E1A proteins are expressed initially, and these proteins induce expression of the “delayed early” proteins encoded by the E1B, E2, E3, and E4 transcription units (reviewed by Shenk, 1996). E1A proteins also induce or repress cellular genes, resulting in stimulation of the cell cycle. About 23 early proteins function to usurp the cell and initiate viral DNA replication.

Viral DNA replicates at about 7 h post-infection (p.i.), then late genes are expressed from the “major late” transcription unit. Major late mRNAs are synthesized from the common “major late promoter” by alternative pre-mRNA processing. Each late mRNA contains a common “tripartite leader” at its 5′-terminus (exons 1, 2 and 3), which allows for efficient translation of Adenovirus late mRNAs. Cellular protein synthesis is shut off, and the cell becomes a factory for making viral proteins.

Virions assemble in the nucleus at about 1 day p.i., and after 2-3 days the cell lyses and releases progeny virus. Cell lysis is mediated by the E3 11.6K protein, also known as ADP (Tollefson et al., 1996b; Tollefson et al., 1996c). The term ADP as used herein in a generic sense refers collectively to ADP's from adenoviruses such as, e.g., Adenovirus type 1 (Ad1), Adenovirus type 2 (Ad2), Adenovirus type 5 (Ad5) or Adenovirus type 6 (Ad6) all of which express homologous ADP's with a high degree of sequence similarity.

Human adenovirus type 5 (Ad5) is particularly useful for cancer gene therapy. It primarily causes asymptomatic or mild respiratory infections in young children, followed by long term effective immunity. Fatalities are extremely rare except when the patient is immunocompromised (Horwitz, 1996). Ad5 is very well understood, can be grown in culture to high titer stocks that are stable, and can replicate in most human cancer cell types (Shenk, 1996). Its genome can be manipulated by site-directed mutagenesis and insertion of foreign sequences.

Adenovirus vectors being investigated for use in anti-cancer and gene therapy are based on recombinant viruses that are either replication-defective or replication-competent. Typical replication-defective Adenovirus vectors lack the E1A and ElB genes (collectively known as E1) and contain in their place an expression cassette consisting of a promoter and pre-mRNA processing signals which drive expression of a foreign gene. The E1A proteins induce transcription of other Adenovirus genes, and in nontransformed cells they deregulate the cell cycle, induce or repress a variety of cellular genes, and force cells from Gₒ into S-phase (White, 1998; Wold et al., 1994). The E1B proteins inhibit cellular apoptosis. Id. These vectors are unable to replicate because they lack the E1A genes required to induce Adenovirus gene expression and DNA replication. In addition, the E3 genes are usually deleted because they are not essential for virus replication in cultured cells.

The inventors have provided a new generation of replication-restricted anti-neoplastic adenovirus vectors (Doronin et al., 2000; 2001; Wold et al., U.S. patent application Ser. No. 09/351,778; Wold et al., PCT/US00/18971; each incorporated herein by references). These vectors may be utilized to express TRAIL proteins in accordance with the present invention. An exemplary vector is named KD3. KD3 has a deletion in the AdE3 transcription unit that removes the genes for the 6.7K, gp19K, RIDα, RIDβ, and 14.7K proteins (Doronin et al., 2000). These E3 genes are believed to protect Ad-infected cells from destruction by killer cells of the immune system. This lack of E3 genes provides a safety feature for the vector because the vector should be more easily controlled by the immune system than wild-type Ad5 (Doronin et al., 2000; U.S. patent application Ser. No. 09/351,778). KD3 has the gene for the ADP protein placed in the E3 region such that ADP is synthesized in much greater amounts and at earlier stages of infection (i.e., ADP is overexpressed relative to wild-type) than in a control virus designated dl1101/1107 (dl101/07) (Doronin et al., 2000).

As a result of overexpression of ADP, KD3 lyses cells more readily and spreads from cell-to-cell more efficiently than dl1101/1107. KD3 also has a feature that prevents KD3 from replicating well in normal cells, yet allows it to replicate in most cancer cell types. This feature is a mutation in the EIA gene that abolishes binding of E1A proteins to the cellular proteins named p300 and pRB. p300 is a transcriptional co-activator and pRB is a tumor suppressor. Since the E1A proteins of KD3 cannot bind and inactivate p300 and pRB, KD3 is unable to deregulate cell cycle and therefore it does not replicate well in normal cells. KD3 does, however, replicate in cancer cell lines inasmuch as the cell cycle is deregulated in such cells. The inventors also have constructed a vector named VRX-007. VRX-007 is exactly like KD3 except it has a wild-type EIA gene. Because VRX-007 overexpresses ADP, it lyses cells more efficiently and it spreads from cell-to-cell more efficiently than Ad5 or a similar control Adenovirus named dl309. Depending on the context, one may select the appropriate viral elements to achieve the particular therapeutic goal.

### III. TRAIL

Tumor Necrosis Factor (TNF)-Related Apoptosis-Inducing Ligand (TRAIL) selectively kills tumor cells (Wiley et al., 1995). TRAIL is cytotoxic to a wide range of tumor cell lines, while most normal cells are resistant to TRAIL treatment (Ashkenazi et al., 1999; Walczak et al., 1999). This specificity may be partly due to the specific expression of decoy receptors 1 and 2 (DcR1, DcR2) on the surface of normal cells (reviewed in Sheridan et al., 1997), and partly due to the anchorage independent growth pattern of tumor cells (Goldberg et al., 2001). The mechanistic details of anchorage independent growth on TRAIL susceptibility remains to be elucidated. Both membrane-bound and secreted forms of TRAIL are cytotoxic to tumor cells. TRAIL administered intraperitoneally (i.p.) or intravenously (i.v.) retarded the growth of xenotransplanted tumors in immunodeficient mice and in some cases caused them to regress (Ashkenazi et al., 1999; Walczak et al., 1999). Importantly, the mice suffered no significant side effects (Ashkenazi et al., 1999).

The protein and nucleic acid sequences for human TRAIL are found in Accession No. U37518 and are represented herein as SEQ ID NO:2 and SEQ ID NO:1 respectively. The protein sequence is set forth below:

| | |
|---|---|
| MAMMEVQGGPSLGQTCVLIVIFTVLLQSLCVAVTYV | (SEQ ID NO: 2) |
| YFTNELKQMQDKYSKSGIACFLKEDDSYWDPNDEES | |
| MNSPCWQVKWQLRQLVRKMILRTSEETISTVQEKQQ | |
| NISPLVRERGPQRVAAHITGTRGRSNTLSSPNSKNE | |
| KALGRKINSWESSRSGHSFLSNLHLRNGELVIHEKG | |
| FYYIYSQTYFRFQEEIKENTKNDKQMVQYIYKYTSY | |
| PDPILLMKSARNSCWSKDAEYGLYSIYQGGIFELKE | |
| NDRIFVSVTNEHLIDMDHEASFFGAFLVG | |

### IV. PROMOTERS AND HIGH LEVEL EXPRESSION

In one embodiment of the present invention, expression of TRAIL and/or ADP is achieved by placing coding regions for these proteins under the control of the Adenovirus MLP. While providing high level expression of the upstream coding region, the downstream coding region is not expressed as highly. Thus, in accordance with the present invention, various other promoters may be used to drive the expression of the downstream gene (or the second gene that not placed under the control of the MLP). A number of promoter options are available, as discussed below.

One of the goals of the invention is to provide overexpression of TRAIL and/or ADP. Overexpression of ADP, with regard to ADP expression from wild-type Ad5 virus at 24 hours p.i., may be 1.5-fold, 2-fold, 3-fold, 4-fold, 5-fold, 10-fold or greater. Using ADP expressing at 24 hours p.i. from wild-type Ad5 as the standard, TRAIL expression may also be quantified as 1.5-fold, 2-fold, 3-fold, 4-fold, 5-fold, 6-fold, 7-fold, 8-fold, 9-fold, 10-fold or greater.

Another way to measure TRAIL production is in microgram (pg) of protein per million infected cells. The inventors have achieved levels of 4.-4.5 μg of TRAIL per million infected cells. Apoptosis is inducible by TRAIL at ng/ml quantities in vitro.

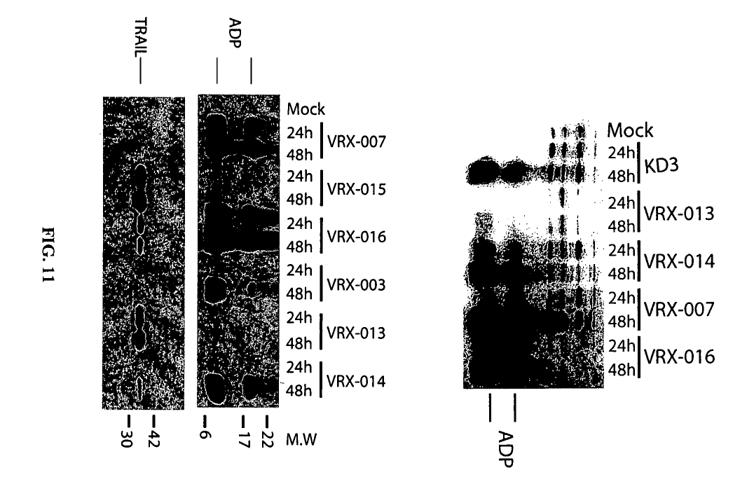

Western blots (FIG. 11, Example 2) suggest that ADP is made in quantities similar to the respective parental viruses for VRX-014 (compared to KD3) and VRX-016 (compared to VRX-007). By extension to previous data, VRX-014 and VRX-016 should therefore express more ADP than dl1101/1107 and Ad5 (which were the respective parental viruses for KD3 and VRX-007).

A. Adenovirus Major Late Promoter

At the onset of DNA replication, the pattern of Adenoviral transcription changes radically from the early to the late genes. There is cis-acting control of this switch, i.e., only newly replicated DNA is used for late gene transcription, but the mechanism controlling this is not understood. Late phase transcription is driven primarily by the major late promoter (MLP). Although transcription from this promoter is complex involving multiple polyadenylation signals and an elaborate usage of RNA splicing, five gene clusters can be defined (L1-L5). Late phase gene expression is primarily concerned with the synthesis of virion proteins. A tripartite leader sequence is found in the 5′ region of the late transcripts. Just upstream of the first splice site is a cap structure, to which 5′GTP is added. Thirty-one base pairs upstream of the promoter is a TATAAAA sequence, but this is not necessary for transcription.

B. Tumor Specific Promoters

Tumor specific promoters may be used in conjunction with an amplifying expression system, described further below. The expression system relies, in the first instance, on the ability of a tissue specific promoter to drive the expression of a transcriptional transactivator, which then turns on a second promoter of interest. In fact, the promoter need not be entirely specific for tumor tissue but, rather, should be active preferentially in tumor tissue. In other words, a small amount of expression in normal tissues, as compared to tumor tissues, may be tolerated. The following tumor specific (or preferential) promoters are contemplated for use in accordance with the present invention.

Carcinoembryonic Antigen (CEA) Promoter. CEA is a membrane glycoprotein that is overexpressed in many carcinomas and is widely used as a clinical tumor marker (Paxton et al., 1987; Thompson et al., 1991). Sequence analysis has identified several molecules that are closely related to CEA, including non-specific cross-reacting antigens (NCA) and biliary glycoprotein (Neumaier et al., 1988; Oikawa et al. 1987; Hinoda et al., 1991). CEA is expressed at low levels in some normal tissues and is usually overexpressed in malignant colon cancers and other cancers of epithelial cell origin. Both CEA and NCA expression is fairly homogenous within metastatic tumors, presumably due to the important functional role of these antigens in metastasis (Robbins et al., 1993; Jessup and Thomas 1989).

The cis-acting sequence that confers expression of the CEA gene on certain cell types has been identified and analyzed (Hauck and Stanners, 1995; Schrewe et al., 1990); Accession Nos. Z21818 and AH003050. It consists of approximately 400 nucleotides upstream from the translational start codon and has sequence homology with a similar sequence in NCA (Schrewe et al., 1990). This promoter has been used to drive some suicide genes and to mediate cell killing in tumor xenografts of stably transfected cells (Osaki et al., 1994; Richards et al., 1995). However, its application in gene therapy is limited by its relatively low transcriptional activity. To solve this problem, Kijima et al. recently used the Cre/loxP system to enhance transgene expression from the CEA promoter (Kijima et al., 1999). In their system, a stuffer DNA flanked by a loxP sequence was placed between a transgene and a strong upstream promoter. For coadministration with a second vector expressing a Cre gene driven by a CEA promoter, the stuffer DNA was removed to permit expression of the transgene from its upstream promoter. However, this approach requires rearrangement of vector molecules and is limited by the transcriptional activity of the upstream promoter which could be weak in some cell types.

hTERT Promoter. Recently, the human telomerase reverse transcriptase (hTERT) has been cloned by several groups and found to be expressed at high levels in primary tumors and cancer cell lines, but repressed in most somatic tissues (Nakamura et al., 1997; Meyerson et al., 1997; Kilian et al., 1997; Harrington et al., 1997). Data suggest that hTERT is a key determinant of telomerase activity. This includes the finding that hTERT expression is highly correlated with telomerase activity and that ectopic expression of hTERT in telomerase-negative cells is sufficient to reconstitute telomerase activity and extend the life span of normal human cells. (Nakamura et al., 1997; Meyerson et al., 1997; Kilian et al., 1997; Harrington et al., 1997; Weinrich et al., 1997; Nakayama et al., 1998; Counter et al., 1998; Bodnar et al., 1998). More recently, it was reported that ectopic expression is required, but not sufficient, for direct tumorigenic conversion of normal human epithelial and fibroblast cells (Hahn et al., 1999).

The promoter region of the hTERT gene also has been cloned (Takakura et al. 1999; Horikawa et al., 1999; Cong et al., 1999); see also Acession Nos. AB016767 and AF097365. The promoter is high G/C (guanine/cytosine)-rich and lacks both TATA and CAAT boxes, but contains binding sites for several transcription factors, including Myc and Spl. Deletion analysis of the hTERT promoter identified a core promoter region of about 200 bp upstream of the transcription start site. Transient assays revealed that he core promoter is significantly activated in cancer cell lines but is repressed in normal primary cells.

PSA Promoter. Prostate specific antigen (PSA) or KLK3 as it is sometimes called, is a serine protease which is synthesized primarily by both normal prostate epithelium and the vast majority of prostate cancers; see Accession No. S81389. The expression of PSA is mainly induced by androgens at the transcriptional level via the androgen receptor (AR). The AR modulates transcription through its interaction with its consensus DNA binding site, GGTACA(n)₃TGTT/CCT, termed the androgen response element (ARE); (Schuur et al., 1996). The core PSA promoter region exhibits low activity and specificity, but inclusion of the PSA enhancer sequence which contains a putative ARE increases expression, specifically in PSA-positive cells. Expression can be further increased when induced with androgens such as dihydrotestosterone (Latham et al., 2000).

AFP Promoter. Alpha-fetoprotein (AFP) is expressed at high levels in the yolk sac and fetal liver and at low levels in the fetal gut; see Accession No. L34019. AFP transcription is dramatically repressed in the liver and gut at birth to levels that are barely detectable by postnatal day 28. This repression is reversible as the AFP gene can be reactivated during liver regeneration and in hepatocellular carcinomas. Previous studies in cultured cells and transgenic mice identified five distinct regions upstream of the AFP gene that control its expression. The promoter and three enhancers functioned as positive regulatory elements, whereas the repressor acted as a negative element. The promoter resides within the 250 bp directly adjacent to exon 1. The repressor, a 600 bp region located between −250 and −850, is required for postnatal AFP repression. Further upstream at −2.5, −5.0 and −6.5 kb are three enhancers termed Enhancer I (EI), EII, and EIII. These three enhancers are active, to varying degrees, in the three tissues where AFP is expressed.

Probasin and ARR2PB promoter. One of the most well-characterized proteins uniquely produced by the prostate and regulated by promoter sequences responding to prostate-specific signals, is the rat probasin protein. Study of the probasin promoter region has identified tissue-specific transcriptional regulation sites, and has yielded a useful promoter sequence for tissue-specific gene expression. The probasin promoter sequence containing bases −426 to +28 of the 5′ untranslated region, has been extensively studied in CAT reporter gene assays (Rennie et al., 1993). Prostate-specific expression in transgenic mouse models using the probasin promoter has been reported (Greenberg et al., 1994). Gene expression levels in these models parallel the sexual maturation of the animals with 70-fold increased gene expression found at the time of puberty (2-6 weeks). The probasin promoter (−426 to +28) has been used to establish the prostate cancer transgenic mouse model that uses the fused probasin promoter-simian virus 40 large T antigen gene for targeted overexpression in the prostate of stable transgenic lines (Greenberg et al., 1995). Thus, this region of the probasin promoter is incorporated into the 3′ LTR U3 region of the RCR vectors thereby providing a replication-competent MoMLV vector targeted by tissue-specific promoter elements.

The probasin promoter confers androgen selectivity over other steroid hormones, and transgenic animal studies have demonstrated that the probasin promoter will target androgen, but not glucocorticoid, regulation in a prostate-specific manner. Previous probasin promoters either targeted low levels of transgene expression or became too large to be conveniently used. Thus, a probasin promoter was designed that would be small, yet target high levels of prostate-specific transgene expression (Andriani et al., 2001). This promoter is ARR2PB which is a derivative of the rat prostate-specific probasin promoter which has been modified to contain two androgen response elements. ARR2PB promoter activity is tightly regulated and highly prostate specific and is responsive to androgens and glucocorticoids.

C. Inducible Promoters

Replication of vectors according to the invention can also be controlled by placing one or more genes essential for vector replication under the control of a promoter that is activated by an exogenous inducing agent, such as metals, hormones, antibiotics, and temperature changes.

Metallothionein promoters. U.S. Pat. No. 4,601,978 describes methods and compositions for controlled expression of genes in mammalian host cells. DNA sequences comprising the human metallothionein II (hMT-II) transcriptional regulatory system, inducible by elevated concentrations of heavy metals and glucocorticoids, includes the promoter region (RNA polymerase recognition and binding sites), the transcriptional initiation sequence (cap site), and the regulatory sequence(s) responsible for inducible transcription. The regulatory system is found on a DNA fragment of fewer than about 500 bp (base pairs) located on the 5′ flanking region of the hMT-II gene upstream of the translational initiation codon. See also U.S. Pat. Nos. 5,089,397 and 6,207,146.

Glucocorticoid promoter. U.S. Pat. No. 5,512,483 discloses a mammalian expression vector containing a synthetic promoter composed of several high affinity glucocorticoid response elements placed upstream of a minimal promoter TATA region. In transiently transfected HeLa cells in the presence of dexamethasone, one of these promoters was at least 50-fold more efficient than the mouse mammary tumor virus long terminal repeat in expressing bacterial chloramphenicol acetyl-transferase (CAT) activity. When the vector was introduced stably into the HeLa cell genome, CAT activity was induced from 10- to more than 50-fold by dexamethasone in 6 of 8 responsive clones. The levels of both basal and induced expression varied from one clone to the next, probably due to an effect of chromosomal location on promoter activity. When propagated stably in HeLa cells in an Epstein-Barr virus episomal vector, the promoter was greater than 50-fold inducible, and its activity was strictly dependent on the presence of dexamethasone. The promoter when stably propagated in HeLa cells was inducible by progesterone in the presence of a transiently transfected progesterone receptor expression vector. These promoters are widely applicable for the strictly controlled high level expression of target genes in eukaryotic cells that contain either the glucocorticoid or progesterone receptors. See also U.S. Pat. Nos. 5,559,027, 5,559,904, and 5,877,018.

Tetracycline response promoter. U.S. Pat. No. 5,464,758 discloses a polynucleotide coding for a transactivator fusion protein comprising the tet repressor and a protein capable of activating transcription in eucaryotes. A second polynucleotide molecule coding for a protein, wherein the polynucleotide is operably linked to a minimal promoter operably linked to at least one tet operator sequence is also disclosed. A method to regulate the expression of a protein coded for by a polynucleotide, by cultivating the eucaryotic cell of the invention in a medium comprising tetracycline or a tetracycline analogue is also disclosed. Kits containing the polynucleotide molecules are also disclosed.

U.S. Pat. No. 5,851,796 discloses a tetracycline-regulated system which provides autoregulatory, inducible gene expression in cultured cells and transgenic animals is described. In the autoregulatory plasmid pTet-tTAk, a modified tTA gene called tTAk was placed under the control of Tetp. Tetracycline prevents tTA from binding to Tetp, preventing expression of both tTA and luciferase. This negative feedback cycle ensures that little or no tTA is produced in the presence of tetracycline, thereby reducing or eliminating possible toxic effects. When tetracycline is removed, however, this strategy predicts that tiny amounts of tTA protein (which may result from the leakiness of the minimal promoter), will bind to Tet-op and stimulate expression of the tTAk gene. A positive feedforward loop is initiated which in turn leads to higher levels of expression of tTA and thus, luciferase. Polynucleotide molecules encoding the autoregulatory system, as well as methods of enhancing or decreasing the expression of desired genes, and kits for carrying out these methods are described. See also U.S. Pat. Nos. 5,971,122, 6,133,027 and 6,440,741.

Heat shock protein (hsp) promoters. The activation and subsequent repression of heat shock genes in Drosophila has been studied by the introduction of cloned segments into Drosophila cells. In particular, the Drosophila hsp70 gene was fused in phase to the E. coli β-galactosidase structural gene, thus allowing the activity of the hybrid gene to be distinguished from the five resident hsp7 heat shock genes in the recipient Drosophila. Drosophila heat shock genes have also been introduced and their activity studied in a variety of heterologous systems, and, in particular, in monkey COS cells (Pelham, 1982; Mirault et al., 1982); and in mouse cells (Corces et al., 1981).

The hybrid hsp70-lacZ gene appeared to be under normal heat shock regulation when integrated into the Drosophila germ line (Lis et al., 1983). Three different sites of integration formed large puffs in response to heat shock. The kinetics of puff formation and regression were exactly the same as those of the 87C locus, the site from which the integrated copy of the hsp70 gene was isolated. The insertion of the 7 kilobase E. coli β-galactosidase DNA fragment into the middle of the hsp70 structural gene appeared to have had no adverse effect on the puffing response. The β-galactosidase activity in the transformants was regulated by heat shock.

Deletion analysis of the Drosophila hsp70 heat shock promoter has identified a sequence upstream from the TATA box which is required for heat shock induction. This sequence contains homology to the analogous sequence in other heat shock genes and a consensus sequence CTxGAAxxTTCxAG has been constructed (Pelham and Bienz, 1982). When synthetic oligonuclueotides, whose sequence was based on that of the consensus sequence, were constructed and placed upstream of the TATA box of the herpes virus thymidine kinase gene (tk) (in place of the normal upstream promoter element), then the resultant recombinant genes were heat-inducible both in monkey COS cells and in Xenopus oocytes. The tk itself is not heat inducible and probably no evolutionary pressure has occurred to make it heat inducible But the facts above indicate that tk can be induced by a heat shock simply by replacing the normal upstream promoter element with a short synthetic sequence which has homology to a heat shock gene promoter.

An inverted repeat sequence upstream of the TATA box is a common feature of many of the heat shock promoters which have been studied (Holmgren et al., 1981). In five of the seven Drosophila promoters, this inverted repeat is centered at the 5′-side of the penultimate A residue of the consensus sequence, but the sequence of the inverted repeat itself is not conserved (Pelham, 1982). In some cases, however, the inverted repeat sequence occurs upstream from the TATA box and the consensus sequence is not present. In these cases, there is no heat inducibility so the presence of the inverted repeat does not substitute for the consensus sequence. See also, U.S. Pat. No. 5,521,284. U.S. Pat. No. 6,649,260 discloses a cold-inducible promoter.

GAL4 promoter. U.S. Pat. No. 5,013,652 describes a DNA expression vector which can be used to express many heterologous proteins at ultrahigh expression levels of no less than 1 gram per liter of yeast culture or at least 10% of total yeast cell protein. A hybrid yeast promoter was composed of elements from two naturally-occurring yeast promoters. The transcription initiation site was derived from the MF-alpha-1 gene. An upstream activation site derived from the regulatory region of the yeast GAL1-10 gene was utilized in place of the MF-alpha-1 upstream activation site. Use of the GAL1-10 upstream activation site permits tightly regulated expression of the MF-alpha-1 transcription initiation site by metabolites such as glucose and galactose.

The GAL4 protein, encoded by the GAL4 gene, is a positive regulatory protein for the yeast galactose system. It has been shown that this protein binds to the GAL1 upstream activation site and is required for high level regulated expression of the 1 gene. Since most mammalian cells express no GAL4-like activity, a synthetic GAL4-responsive promoter containing GAL4-binding sites and a TATA box should have no or extremely low basal activity in the absence of a GAL4 transactivator, and high activity in its presence. The GAL4 transcriptional activator derived from yeast, that when fused to a highly acidic portion of the herpes simplex virus protein VP16, is a very potent activator of transcription (Sadowski et al., 1988). Thus, genes that have GAL4 binding sites in their promoter regions, are highly activated by the introduction of the GAL4-VP16 fusion protein. A synthetic promoter composed of a minimal TATA box and five consensus 17-mer GAL4-binding site elements (GAL4/TATA) has also been described.

Another transcriptional activator that could be used in a similar manner is a GAL4-estrogen receptor fusion protein (GAL4-ER), where the GAL4 protein is fused to the hormone binding region of the human estrogen receptor (Braselmann et al., 1993). It is envisioned that the VP16 protein could also be added to this complex to render the complex more potent and less cell type restricted, as compared to GAL4-ER alone. The estrogen receptor targets the estrogen response element and thus can be used as an independent regulator of transcription initiation.

D. Internal Ribosome Binding Sites

When combining multiple open reading frames in a single transcript, it may prove desirable to include an internal ribosome entry site (IRES). IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

E. Enhancing Expression Level

The present invention further contemplates methods for enhancing the expression of the Adenoviral-TRAIL vectors and thus, improving their therapeutic effect. Enhancing expression of Adenoviral-TRAIL may involve insertion of a splice site or spacer region, or protein—protein interaction domain or motif within the Adenoviral-TRAIL construct. Splice sites, spacer regions and protein-protein interaction domains or motifs are well known in the art as are methods for using them.

In particular embodiments, it is contemplated that a protein—protein interaction domain or motif may be employed to promote and stabilize trimerization of TRAIL thereby further enhancing the biological activity of the Adenoviral-TRAIL vectors. Such a protein—protein interaction domain or motif may be fused to the N-terminal region of TRAIL. Protein—protein interaction domains or motifs are well known to one of ordinary skill in the art. Examples of protein—protein interaction domains or motifs that may be employed in the present invention may include, but are not limited to, a leucine zipper (Walczak et al., 1999; Shu et al., 1999; O'Shea et al., 1989), triple-stranded alpha-helical coiled-coil (Peterandenderl et al., 1992), an isoleucine zipper (Morris et al., 1999), the coiled-coil neck domain of surfactant protein D (SP-D) (McAlinden et al., 2002) or surfactant protein A (SP-A) (Palaniyar et al., 2001), and the carboxyl noncollagenous 1 (NC1) domain of collagen X (Zhang et al., 1999). One may also employ the use of a signal sequence of to directs TRAIL into a target cell.

It is also contemplated that expression of TRAIL in a cell may be enhanced by insertion of a 3′ splice site at the intergene region of the Adenoviral-TRAIL constructs described in Example 1 and shown in FIG. 1. Such constructs, for example, may comprise of ADP followed by the intergene region containing the 3′ splice site and TRAIL, in that order. Similiarly, an alternate construct may comprise TRAIL followed by the intergene region containing the 3′ splice site followed by ADP. In some instances it may be preferred to place both the ADP and the TRAIL genes in the E3 region thereby further enhancing the level of expression of TRAIL in cells. Methodologies for generating such constructs are disclosed herein.

To enhance the expression and therapeutic efficacy of the Adenoviral-TRAIL constructs of the invention it is contemplated that the entire coding sequence of soluble TRAIL (extracellular portion), (Armeanu et al., 2003, Griffith et al., 2001, 2000), rather than the wild-type synthetic TRAIL gene, may be employed in creating a Adenoviral-TRAIL construct.

### V. TREATING SUBJECTS WITH HYPERPROLIFERATIVE DISORDERS

Thus, in accordance with the present invention, a patient suffering from a hyperproliferative disorder may be treated with an appropriate vector. A major hyperproliferative disease is, of course, cancer. Any number of cancers may be treated, for example, brain cancer, head and neck cancer, esophageal cancer, lung cancer, thyroid cancer, stomach cancer, colon cancer, liver cancer, kidney cancer, prostate cancer, breast cancer, cervical cancer, ovarian cancer, testicular cancer, rectal cancer, skin cancer or blood cancer. Also contemplated are benign disorders such as benign prostatic hyperplasia, restenosis, primary psoriasis, angiogenesis, rheumatoid arthritis, inflammatory bowel disease, psoriasis, eczema, secondary cataracts, or bronchial dysplasia. As discussed below, the constructs and methods of delivery may vary and can be used as appropriate.

A. Pharmaceutical Formulations & Routes of Administration

Pharmaceutical compositions of the present invention comprise an effective amount of one or more adenoviral particles dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one vector or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, intramuscularly, subcutaneously, subconjunctivally, intravesicularly, mucosally, intrapericardially, intraumbilically, intraoculally, orally, topically, locally, by inhalation (e.g., aerosol inhalation), by injection, by infusion, by continuous infusion, localized perfusion bathing target cells directly, via a catheter, via a lavage, in cremes, or by other method or any combination of the forgoing as would be known to one of ordinary skill in the art (see, for example, Remington's Pharmaceutical Sciences, 18th Ed. Mack Printing Company, 1990, incorporated herein by reference). Of particular interest is delivery local or regional to a tumor site, circumferential treatment of a tumor site, and treatment of post-operative tumor bed, including catheterization of a body cavity.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. In particular embodiments, prolonged absorption of an injectable composition can be brought about by the use in the compositions of agents delaying absorption, such as, for example, aluminum monostearate, gelatin or combinations thereof.

B. Combination Therapies

In order to increase the effectiveness of a therapy according to the present invention, it may be desirable to combine these Adenoviral-TRAIL vectors with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, overcoming drug or multidrug resistance, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Adenoviral-TRAIL may be provided at the same time with the secondary therapy. Alternatively, the Adenoviral-TRAIL therapy may precede or follow the other agent treatment by intervals ranging from minutes to weeks. In embodiments where the other agent and Adenovirus are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, Adenoviral-TRAIL therapy is “A” and the secondary agent, such as radio- or chemotherapy, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

1. Chemotherapy

Cancer therapies also include a variety of combination therapies with both chemical and radiation based treatments. Chemotherapeutic agents contemplated for use in combination with Adenoviral-TRAIL therapy include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxol, gemcitabien, navelbine, farnesyl-protein transferase inhibitors, transplatinum, 5-fluorouracil, floxuridine, mutamycin, vincristin, vinblastin and methotrexate, or any analog or derivative variant of the foregoing.

2. Radiotherapy The present invention also contemplates the use of other factors that cause DNA damage that have been used extensively in the art in combination with Adenoviral-TRAIL therapy. These damaging factors include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Immunotherapy

Immunotherapeutics, generally, rely on the use of immune effector cells and molecules to target and destroy cancer cells. Thus, the present invention contemplates the use of immunotherapeutic agents in combination with Adenoviral-TRAIL for treating cancer therapy. The immune effector may be, for example, an antibody specific for some marker on the surface of a tumor cell. The antibody alone may serve as an effector of therapy or it may recruit other cells to actually effect cell killing. The antibody may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

4. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In accordance with the present invention, one may combine Ad-TRAIL therapy with various other gene therapies. Therapeutic polypeptides are described below.

Tumor Suppressors. The tumor suppressor oncogenes function to inhibit excessive cellular proliferation. The inactivation of these genes destroys their inhibitory activity, resulting in unregulated proliferation. The tumor suppressors p53, Rb and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with viral proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Inducers of Apoptosis. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{w}, Bclₛ, Mcl-1, A1, Bfl-1) or counteract Bcl-2 function and promote cell death (e.g., Bax, Bak, Bik, Bim, Bid, Bad, Harakiri).

Inducers of Cellular Proliferation. The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that antisense or ribozyme construct directed to a particular inducer of cellular proliferation is used to prevent expression of the inducer of cellular proliferation.

The proteins FMS, ErbA, ErbB and Neu are growth factor receptors. Mutations to these receptors result in loss of regulatable function. For example, a point mutation affecting the transmembrane domain of the Neu receptor protein results in the Neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc also are proteins that directly exert their effects on nuclear functions as transcription factors. An extensive list of oncogenes that could be the targets for antisense therapy is present below.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymidine (A:T) in the case of DNA, or adenine paired with uracil (A:U) in the case of RNA. Inclusion of less common bases such as inosine, 5-methylcytosing, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

Particular oncogenes that are targets for antisense constructs are ras, myc, neu, raf, erb, src, fms, jun, trk, ret, hst, gsp, bcl-2 and abl. Also contemplated to be useful will be anti-apoptotic genes and angiogenesis promoters.

Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). Ribozyme catalysis has primarily been observed as part of sequence-specific cleavage/ligation reactions involving nucleic acids (Joyce, 1989). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme. Targets for this embodiment will include angiogenic genes such as VEGFs and angiopoietins as well as the oncogenes (e.g., ras, myc, neu, raf, erb, src, fms, jun, trk, ret, hst, gsp, bcl-2, EGFR, grb2 and abl).

interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity. (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et. al. wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et. al., 2000; Elbashir et. al., 2001). WO 99/32619 and WO 01/68836 suggest that RNA for use in siRNA may be chemically or enzymatically synthesized. Both of these texts are incorporated herein in their entirety by reference. Similarly, WO 00/44914, incorporated herein by reference, suggests that single strands of RNA can be produced enzymatically or by partial/total organic synthesis. WO 01/36646, incorporated herein by reference, places no limitation upon the manner in which the siRNA is synthesized, providing that the RNA may be synthesized in vitro or in vivo, using manual and/or automated procedures.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which are equipped at each end with a promoter sequence. The templates are preferably attached to a solid surface. After transcription with RNA polymerase, the resulting dsRNA fragments may be used for detecting and/or assaying nucleic acid target sequences.

Cytokines. Another class of genes that is contemplated to be inserted into the adenoviral vectors of the present invention include interleukins and cytokines. Interleukin 1 (IL-1), IL-2, IL-3, IL-4, IL-5, IL-6, IL-7, IL-8, IL-9, IL-10, , IL-11, IL-13, IL-13, IL-14, IL-15, β-interferon, α-interferon, γ-interferon, angiostatin, thrombospondin, endostatin, METH-1, METH-2, GM-CSF, G-CSF, M-CSF and tumor necrosis factor.

Toxins. Various toxins are also contemplated to be useful as part of the expression vectors of the present invention, these toxins include bacterial toxins such as ricin A-chain (Burbage, 1997), diphtheria toxin A (Massuda et al., 1997; Lidor et al., 1997), pertussis toxin A subunit, E. coli enterotoxin toxin A subunit, cholera toxin A subunit and pseudomonas toxin c-terminal. It has been demonstrated that transfection of a plasmid containing the fusion protein regulatable diphtheria toxin A chain gene was cytotoxic for cancer cells. Thus, gene transfer of regulated toxin genes might also be applied to the treatment of cancers (Massuda et al., 1997).

Single Chain Antibodies. In yet another embodiment, one gene may comprise a single-chain antibody. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule.

Single-chain antibody variable fragments (scFvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al., 1990). These Fvs lack the constant regions (Fc) present in the heavy and light chains of the native antibody.

Antibodies to a wide variety of molecules are contemplated, such as oncogenes, growth factors, hormones, enzymes, transcription factors or receptors. Also contemplated are secreted antibodies, targeted to serum, against angiogenic factors (VEGF/VSP; βFGF; αFGF) and endothelial antigens necessary for angiogenesis (i.e., V3 integrin). Specifically contemplated are growth factors such as transforming growth factor and platelet derived growth factor.

Transcription Factors and Regulators. Another class of genes that can be applied in an advantageous combination are transcription factors. Examples include C/EBPα, IκB, NFκB, Par-4 and C/EBPα.

Cell Cycle Regulators. Cell cycle regulators provide possible advantages, when combined with other genes. An example of a regulator that serves to inhibit cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4}, which has been biochemically characterized as a protein that specifically binds to and inhibits CDK4, and thus may regulate Rb phosphorylation (Serrano et al., 1993; Serrano et al., 1995). Since the

16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK}4 belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Okamoto et al., 1994; Nobori et al., 1995; Orlow et al., 1999). Restoration of wild-type p16^{INK4} function by transfection with a plasmid expression vector reduced colony formation by some human cancer cell lines (Okamoto, 1994).

Other such cell cycle regulators include p27, p21, p57, p18, p73, p19, p15, E2F-1, E2F-2, E2F-3, p107, p130 and E2F-4. Other cell cycle regulators include anti-angiogenic proteins, such as soluble Flt1 (dominant negative soluble VEGF receptor), soluble Wnt receptors, soluble Tie2/Tek receptor, soluble hemopexin domain of matrix metalloprotease 2 and soluble receptors of other angiogenic cytokines (e.g. VEGFR1/KDR, VEGFR3/Flt4, both VEGF receptors).

Chemokines. Genes that code for chemokines also may be used in the present invention. Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Such chemokines include RANTES, MCAF, MIP1-alpha, MIP1-Beta, and IP-10. The skilled artisan will recognize that certain cytokines are also known to have chemoattractant effects and could also be classified under the term chemokines.

Other Agents. It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cells. Human cancer cell lines A549 (human lung carcinoma), H441 (papillary lung carcinoma), Hep3B (human hepatocellular carcinoma), HepG2 (human hepatoblastoma), SW1116, LS513, LS174T, SW480 (human colon cancer), DLD-1 (colorectal carcinoma), and LNCaP (prostate cancer), were obtained from the American Type Culture Collection. HeLa (cervical carcinoma) cells were from Eileen White (Rutgers University), and KB cells were from Maurice Green (St. Louis University). HT29.14S cells were obtained from Jeff Browning (Biogen, Cambridge, Mass.). HEK 293 cells were obtained from Microbix (Toronto, Ontario, Canada).

Considering that TRAIL induces apoptosis, the inventors constructed a cell line that is resistant to TRAIL-induced apoptosis in order to facilitate the development of the TRAIL-expressing Adenovirus vectors. 293 cells were transfected with pCDNA3-CrmA plasmid (kindly provided by David Pickup, Duke University) and were selected with G418 (400 μg/ml). The outgrowing colonies were isolated and further propagated in G418. The progeny of 12 colonies were tested in a western blot and an immunofluorescence assay using an anti-CrmA antiserum. The culture with the highest number of CrmA expressing cells was subcloned, and the procedure above was repeated, yielding the 293CrmA cell line. This cell line is homogenous for high level CrmA expression. 293CrmA cells were then transfected with p181, a plasmid that expresses all E3 proteins except ADP from the CMV promoter (Toth et al., 2002), and selected with Zeocin (200 μg/ml). The cloning procedure described above was repeated. Seventeen progeny cultures were tested in an immunofluorescence assay for expression of the Adenovirus E3-coded proteins named gp19K, RIDα, RIDβ, and 14.7K. Cultures with the highest levels of these E3 proteins were tested in a western blot for the presence of the RIDα, RIDβ, and 6.7K proteins. The cell line expressing the highest levels of those proteins was named 293crmAE3.

A549, Hep3B, HeLa, 293, 293crmA and 293crmAE3 cells were grown in Dulbecco's modified essential medium (DMEM) supplemented with 10% fetal bovine serum (FBS). LS513, LS 174T, H441 and LNCaP cells were grown in RPMI medium 1640 (HyClone, Logan, Utah) plus 10% FBS. HT29.14S cells were grown in McCoy's 5A medium (Gibco/BRL, Carsbad, Calif.) plus 10% FBS. HepG2 cells were grown in F12-DME (10% FBS), SW1116 and SW480 were grown in Leibowitz's L15 medium, supplemented with 10% FBS.

Viruses. Ad5 mutants dl309 (Jones and Shenk, 1979) and dl1101/1107 (obtained from Stanley Bayley, McMaster University) were described previously. Mutant dl1101/1107 has the same two small deletions in E1A as does dl01/07/520, an E1A mutant that is defective in inducing DNA synthesis in primary rat kidney cells (Howe et al., 1990). However, dl1101/1107 expresses both 13S and 12S E1A mRNAs whereas dl01/07/520 expresses only the 12S E1A mRNA. The Adenovirus KD3 vector, which overexpresses ADP and is restricted to tumor cells by mutations in the E1A region, has been previously described (Doronin et al., 2000). The E1A deletions in KD3 were derived from dl1101/1107.

Adenovirus VRX-013 is a modification of KD3 that has the full-length human TRAIL cDNA inserted into a unique XbaI site (bp 28592 in Ad5) just upstream of adp in the KD3 virus. To construct VRX-013, a SwaI-SgrI B fragment (containing the full length TRAIL cDNA) of pGT60hTRAIL (GIBCO/Invitrogen, Carlsbad, Calif.) was blunt-ended and cloned into the unique SmaI site in pBluescript SK(+) (Stratagene, La Jolla, Calif.). The XbaI-ClaI B fragment of the resulting p591 plasmid was blunt-ended and cloned into the blunt-ended XbaI site just upstream of adp in plasmid pKD3 (Doronin et al., 2000). This resulting plasmid pJW114 as well as dl1101/1107 DNA that had been digested with EcoRI and Spel were cotransfected into 293crmAE3 cells using the calcium phosphate technique. Two plaques were obtained, and these were screened by PCR and restriction enzyme digestion for the presence of recombinant versions of the TRAIL cDNA and adp. These two plaques were each purified three times on 293crmAE3 cells and expanded into large-scale CsCl stocks in KB cells (Tollefson et al., 1998). The titer was determined on 293crmA cells. The viruses resulting from these two plaques are named VRX-013#7 and VRX-013#8. The VRX-013#7 virus stock was used in this document unless otherwise indicated and is referred to as VRX-013.

The virus vectors named VRX-014, VRX-015, VRX-016 were constructed as follows. Shuttle plasmid pL2L1 contains sequences of the Ad5 genome from 60-100 map units with the E3 region deleted (−Ad5 bp 28598 to 30469-) and replaced with an XbaI site at the deletion. A PCR fragment of the human TRAIL open reading frame (ORF) with flanking XbaI sites was cloned into the XbaI site in pL2L1. Primers were designed such that the downstream XbaI site of TRAIL contained a methylation sequence, so the upstream XbaI site would be used for further cloning. Next, a PCR fragment with XbaI sites flanking the linker region between TRAIL and ADP in VRX-013 was cloned into the XbaI site upstream of TRAIL and a PCR fragment comprising Ad5 bp 29497 to 29783 (containing the ADP ORF) and flanking SpeI sites was cloned into the XbaI site upstream of linker region. The resulting plasmid pL2/ADP-linker-TRAIL was co-transfected into 293crmAE3 cells along with dl1101/1107 or dl327 virion DNA digested with EcoRI to make VRX-014 or VRX-016. The plasmid JW114 (TRAIL-linker-ADP) was transfected into 293crmAE3 cells along with VRX-007 virion DNA digested with EcoRI/SpeI to make VRX-015. The resulting plaques for virus vectors VRX-014 and VRX-016 were screened for the expected genome structure and were plaque purified at least two times on 293crmAE3 cells. Plaques have also been obtained that are expected to be the VRX-015 vector, but these plaques have not yet been confirmed by restriction endonuclease digestion and sequencing of the genomic DNA to be VRX-015.

Cytotoxicity. For comparative cytotoxicity determination of VRX-013, KD3 and dl309 on different cancer cell lines, cells were infected at 10 PFU/cell. At 5 days postinfection (p.i.) (SW1116, LS513, SW480, HepG2), 7 days p.i. (LS174T), or 8 days p.i. (LNCaP) medium was removed and attached cells were trypsinized and pooled with cells in the supernatant. Viable and nonviable cell counts (400-700 total) were determined by trypan blue exclusion.

Western blots. Cell monolayers were infected with 10-50 plaque-forming units (PFU) of KD3, VRX-007, VRX-013, VRX-014, or VRX-016 per cell. At 24-48 h p.i., the supernatants were collected and the cells were washed two times with phosphate-buffed saline (PBS) and harvested by scraping into 1 ml PBS. For characterization of 293crmA and 293crmAE3 cell lines, cells in 60 mm dishes were used and processed as above but without infection. The pelleted cells were lysed in lysis buffer (25 mM Tris-Cl pH 8.0, 150 mM NaCl, 0.5% sodium deoxycholate, 0.1% sodium dodecyl sulfate [SDS], and 1× protease inhibitor mix (Boehringer Mannheim, Mannheim, Germany). The protein concentration was measured using the Bio-Rad DC protein assay kit (Bio-Rad Laboratories, Hercules, Calif.), and 10 μg of each sample were electrophoresed on sodium dodecyl sulfate polyacrylamide gels. The gels were electroblotted onto polyvinylidene difluoride membranes (Immobilon; Millipore, Bedford, Mass.). The membranes were blocked in TBST (50 mM Tris-Cl [pH 7.6], 150 mM NaCl, 0.2% Tween 20) containing 10% dry milk (Carnation) overnight at 4° C. After blocking, the membranes were incubated with different primary antibodies depending on the experiment; these antibodies were a rabbit polyclonal antiserum against ADP (Tollefson et al., 1992), a rabbit polyclonal antiserum against human TRAIL (#500-P135; Peprotech, Rocky Hill, N.J.), an anti-RIDβ polyclonal antibody raised against amino acids 118-132 of RIDβ (Tollefson, 1990), an antibody against crmA (kindly provided by David Pickup, Duke University), or with M73, a monoclonal antibody against E1A (Harlow et al., 1985). The secondary antibodies were goat anti-rabbit immunoglobulin G (IgG)-horseradish peroxidase or goat anti-mouse IgG-horseradish peroxidase. The blots were developed using the ECL protocol (Amersham Pharmacia, Arlington Heights, Ill.).

Titration of secreted TRAIL and kinetics of cytotoxicity. 293 and A549 cells (in 60 mm dishes) were infected with 50 PFU/cell of KD3 or VRX-013. At 24 h p.i., supernatants were removed. Supernatants in serial dilutions were used to treat A549 cells in 96-well plates (2×10⁴ cells/well). After 24 h, viability was determined using the MTT assay (Krajcsi et al., 1996). The 3.2:1 dilution was to examine the kinetics of cytotoxicity on A549 cells in 96-well plates (2×10⁴ cells/well). At 24 h post-treatment, viability was determined using the MTT assay (Krajcsi et al., 1996).

Vector spread assay. Cells were seeded in 48-well plates and were mock-infected or infected with 10-fold serial dilutions of the indicated viruses, ranging from 10¹ to 10⁻⁴ PFU/cell. Monolayers were fixed and stained with crystal violet at various days p.i., as described previously (Doronin et al., 2000).

Vector-induced cytopathic effect. Monolayers of A549 cells were grown in 60 mm dishes with 5 ml of DMEM (10% FBS) and were mock-infected or infected with VRX-013 or KD3 at multiplicities of infection ranging from 10 to 10⁻³ PFU/cell. Phase-contrast images of monolayers were taken at 48 h p.i. using a Nikon camera attached to a Nikon TMS phase-contrast microscope.

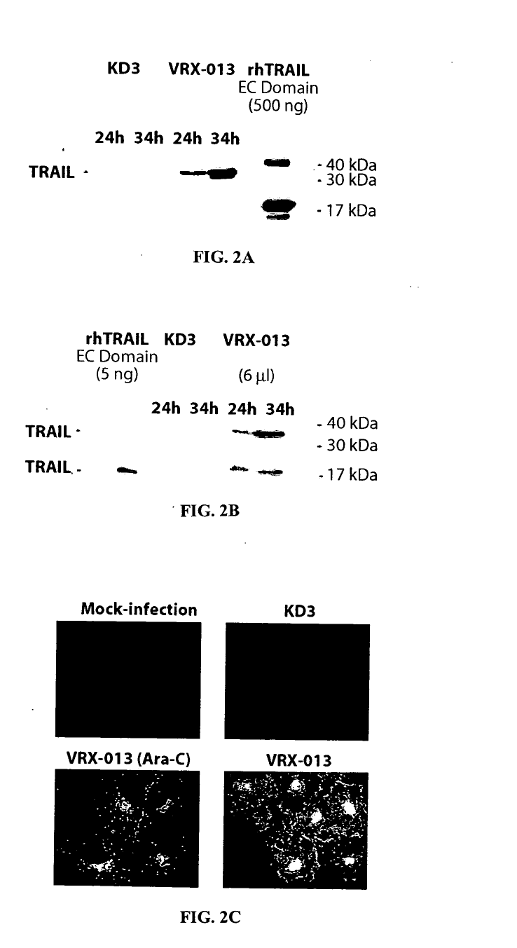

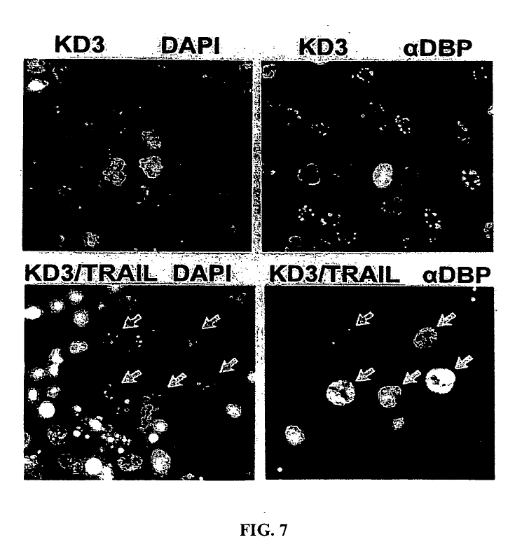

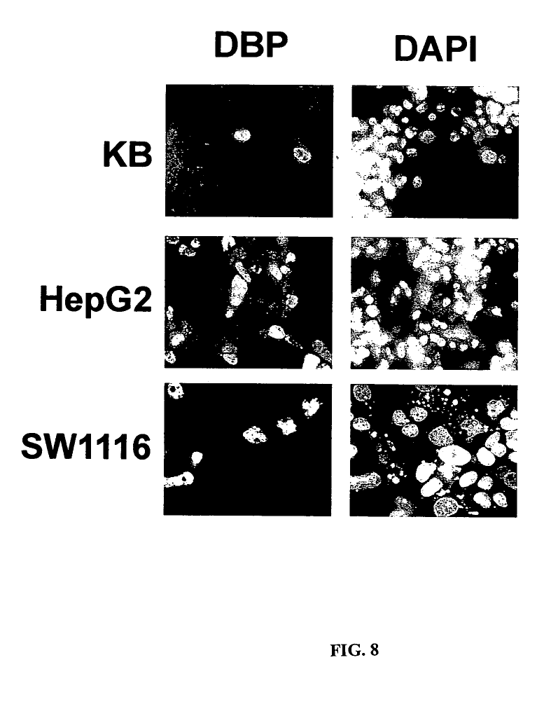

Immunofluorescence. In the experiment shown in FIG. 2C, A549 cells were plated on Coming No. 1 coverslips in 35-mm dishes, and were mock-infected or infected with KD3 or VRX-013 at 10 PFU/cell. At 7 h p.i. VRX-013 infected cells in one of the dishes were treated with 1-β-D-arobinofuranosylcytosine (ara-C) (Sigma, St. Louis, Mo.) at 20 μg/ml. At 23 h p.i. the cells were fixed in methanol (−20° C.) and immunostained for TRAIL using the rabbit polyclonal antiserum against human TRAIL (#500-P135; Peprotech, Rocky Hill, N.J.). In the experiment shown in FIG. 7, A549 cells were infected with 10⁻² PFU/cell of VRX-013 or KD3. At 4 days p.i. cells were fixed in methanol (−20° C.) containing DAPI, then immunostained for the Adenovirus E2-coded DNA-binding protein (DBP) using a rabbit anti-peptide antiserum specific for the C-terminus of DBP (kind gift from Maurice Green, St. Louis University). For FIG. 7, photographs were taken on a Nikon epifluorescence microscope using a 100× Planapo lens and Tmax 400 film (Kodak). The film was developed in Diafine developer. For FIG. 8., cells were infected with VRX-013 at low MOI: SW1116 (1.0 PFU/cell), KB (0.1 PFU/cell), and Hep GZ (10. PFU/cell). At 2 d p.i. cells were fixed and stained with DAPI, then immunostained for DBP. Images were taken on a Nikon epifluorescence microscope using a Nikon DXM 1200 digital camera and ACT-1 software (Nikon Instruments, Inc., Melville, N.Y.).

In vivo anti-tumor efficacy. To test anti-tumor potential of VRX-013, subcutaneous Hep3B xenografts were established in nude mice. Three weeks later, established tumors of average size of 300 mm³ were injected intratumorally with 5×10⁹ PFU/injection of KD3, VRX-013, or vehicle. Injections were repeated 5 times at 2 day intervals (total dose 2.5×10¹⁰ PFU/tumor). Measurements were taken with digital calipers and data were analyzed using the “Mouser” computer program. Tumor volumes were calculated with the following formula: length×width².

### Example 2

### Results

Construction of VRX-013. Plasmid pJW114 was constructed by inserting the full-length copy of the human TRAIL cDNA into the unique XbaI site (position 28592 in Ad5) in plasmid pKD3 (Doronin et al., 2000). The resulting plasmid has three genes in the E3 transcription unit, the Ad5 adp, the Ad5 12.5K, and trail.

Plasmid pJW114 and EcoRI-SpeI-digested dl1101/1107 (Doronin et al., 2000) DNA were cotransfected into 293crmAE3 cells. Following transfection, the complete genome of VRX-013 was formed by overlap recombination of the EcoRI-SpeI-A fragment of dl1101/1107 and plasmid pJW114. The scheme of the resulting virus, VRX-013 is shown in FIG. 1A. The 293crmAE3 cell line treated with VRX-013 showed successful results. This result is in contrast to that of another research group which reported the construction of a replication-defective Adenovirus vector expressing TRAIL from a constitutive promoter (Griffith et al., 2000). The result of this study may differ from those of Griffith et al. (2000) because the vectors of the present invention probably express more TRAIL than their vector. The 293CrmAE3 cells are stably transfected with the poxvirus CrmA gene, the Adenovirus RIDα gene, and the Adenovirus RIDβ gene. CrmA and the RIDα plus RIDβ complex are independent inhibitors of apoptosis. As shown in the western blot in FIG. 1B, the 293CrmAE33 cells do in fact express the CrmA and RIDβ proteins.

VRX-013 expresses a large amount of TRAIL. To characterize TRAIL expression by VRX-013, A549 cells were infected with 50 PFU/cell of vector and at different time points supernatants were collected. Cells were lysed and samples corresponding to equal amounts of total proteins were analyzed by immunoblotting. As shown in FIG. 2A, a form of TRAIL was obtained from all lysates that migrated as a 34-36 kDa band. This band corresponds to the membrane-bound form of the protein (Mariani and Krammer, 1998; Bodmer et al., 2000). Two distinct forms of TRAIL were found in the supernatant (FIG. 2B). The molecular mass of the smaller ca. 19-20 kDa band is similar to the size of the extracellular domain (Mariani and Krammer, 1998). The larger band is most likely the membrane bound form, associated with the apoptotic vesicles in the supernatant. As anticipated, VRX-013 expressed large amount of TRAIL. The secreted form was present at around 800-900 ng/ml (167×5 ng/ml). This adds up to 4-4.5 μg/million cells. With the larger, membrane-bound form the total amount was in the low μg/ml range at 34 h. Importantly, there was a significant amount of TRAIL expressed at 24 h p.i.

Further evidence for TRAIL synthesis, transport, and kinetics of expression came from immunofluorescence studies (FIG. 2C). Mock- or KD3-infected A549 cells did not express detectable TRAIL at 23 h p.i. Cells infected with VRX-013 expressed large amounts of the TRAIL protein. When infection was limited to the early phase by the addition of Ara-C (an inhibitor of adenovirus DNA replication which prevents the transition from the early to late state of infection), the amount of TRAIL was much less than in the late phase of infection (FIG. 2C). It is highly likely that at the early phase of infection the TRAIL gene is transcribed from the E3 promoter, while at late phase, it is probably expressed as part of the major late transcription unit. As shown by Tollefson et al. (1992), ADP expression follows a similar pattern, and it has been proposed that adp is part of the early as well as the late transcription unit. Both at the early, and in particular at the late phase of expression, clear surface staining is visible showing normal transport of the TRAIL protein.

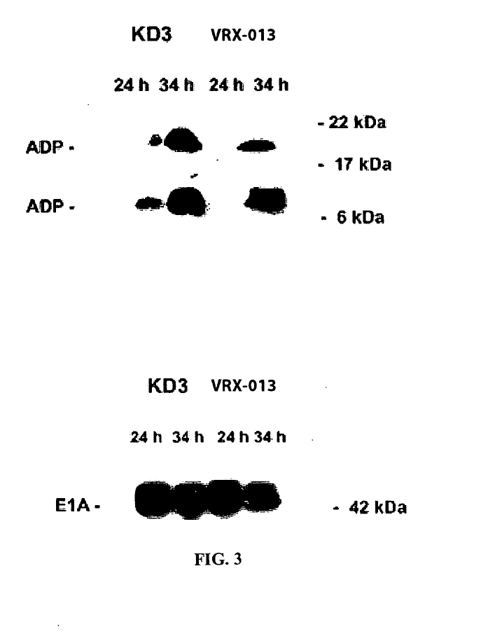

To determine how insertion of the trail gene just upstream of the adp gene affected gene expression of the latter, a comparative analysis of the kinetics of expression of ADP from KD3 versus VRX-013 was performed. ADP migrates as two groups of bands. The upper bands are the glycosylated forms, and the lower bands are proteolytic processing products (Scaria et al., 1992). Two distinctive differences were found in the ADP expression pattern in the KD3 vs VRX-013 infected cells. First, the expression of ADP in VRX-013-infected A549 cells was delayed as compared to ADP expression in KD3-infected cells (FIG. 3A). Second, the upper(glycosylated)-to-lower(non-glycosylated) band ratio in the VRX-013-infected cells was lower than in the KD3-infected cells (FIG. 3A). KD3 and VRX-013 expressed similar amounts of the E1A protein to each other at 24 h and 34 h p.i. (FIG. 3B), indicating that the infections were equivalent and that TRAIL expression and decreased levels of ADP do not markedly affect synthesis of the E1A protein.

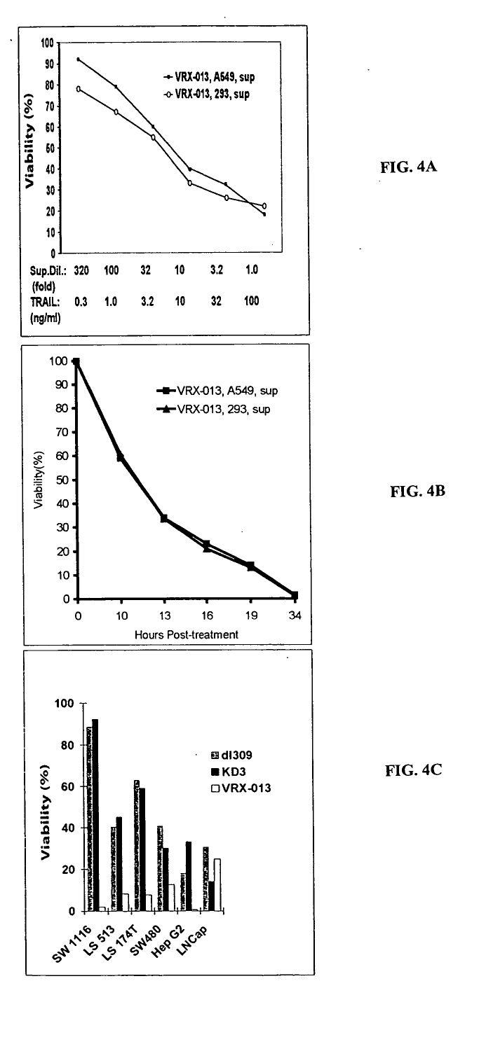

Supernatants from VRX-013-infected cells are highly cytotoxic due to the high TRAIL content. To examine whether supernatants from VRX-013-infected A549 and 293 cells were cytotoxic due to their TRAIL content, A549 and 293 cells were infected with 50 PFU/cell of VRX-013 or KD3. At 28 h p.i., supernatants were collected and used to treat A549 cells. At 24 h post-treatment, cell viability was determined with the MTT assay. Undiluted supematants from the VRX-013-infected cells were highly cytotoxic, inducing 88% apoptosis of A549 cells (FIG. 4A). When the supernatants were serially diluted to as low as 320:1, the amount of cell death correspondingly decreased. In the cells treated with the supernatant from KD3-infected cells, cell death was less than <10% (data not shown), strongly suggesting that the cytotoxicity observed with the VRX-013 supernatants was due to TRAIL. The cytotoxicity of the VRX-013 supernatant was almost certainly not due to the virus present in the supernatant, since at 28 h p.i. viral cell lysis was extremely low (Tollefson et al., 1996).

The kinetics of the cell death were also studied using the 3.2:1 dilution of the supernatants from the VRX-013-infected A549 and 293 cells. These supernatants were added to A549 cells and cell viability was determined at different time-points p.i. The supernatants killed A549 cells with identical kinetics, with cell death apparent at 10 h post-treatment and complete by 34 h (FIG. 4B).

VRX-013 was significantly more cytotoxic than either dl309 or KD3 on various cancer cells lines (FIG. 4C). The fact that KD3 was more potent in killing LNCaP cells, known for TRAIL resistance (Nesterov et al., 2001), provides evidence that TRAIL confers the increased cytotoxicity of VRX-013. Thus it appears that cells infected with VRX-013 secrete large amounts of TRAIL that is functionally active in cytotoxic assays.

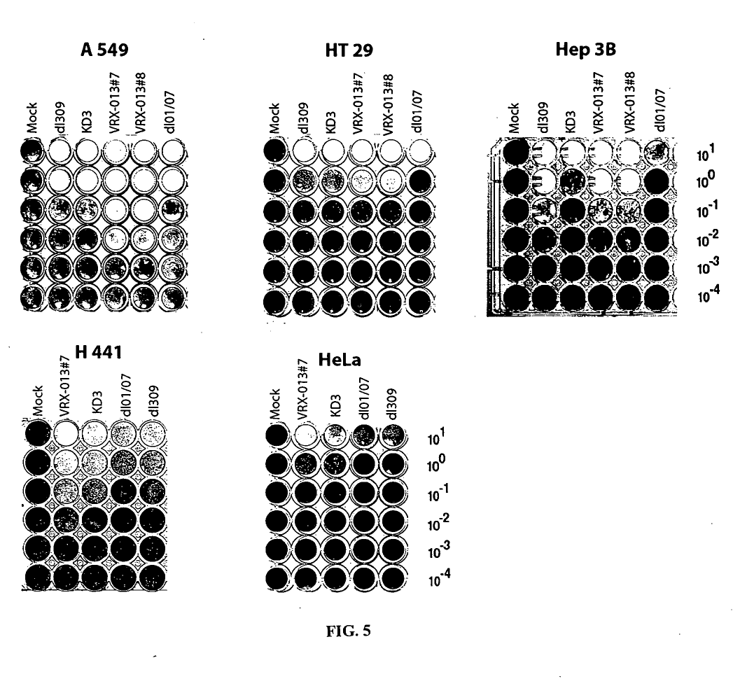

VRX-013 induces much more cell death than KD3. The ability of VRX-013 and KD3 to spread from cell-to-cell was measured in a “vector spread” assay (Doronin et al., 2000). A549, H441, and HeLa cells were mock-infected or infected with VRX-013, KD3, dl1101/1107 (01/07), or dl309 (309) at multiplicities of infection (MOIs) ranging from 10 to 10⁻⁴ PFU/cell (VRX-013 from both plaques 7 and 8 were used in the assay). At 7 days p.i., cells still adhering to the plates were stained with crystal violet. VRX-013 induced much more rapid cell killing (i.e., cell detachment) at 10 PFU/cell in all cell lines and at 1 PFU/cell in A549 and H441 cells. KD3 got off to a slower start but partly made up for the delay in later stages of the assay. VRX-013 was more efficient by more than 1 log in killing A549 and H441 cells than were KD3, dl1101/1107 and dl309 (FIG. 5). All viruses were much less efficient in killing HeLa cells and HT29 cells (FIG. 5), but VRX-013 appeared to be superior in killing even in these cell lines. Hep3B cells were an intermediate phenotype: VRX-013 was more than a log more potent than KD3 but only about equally as potent as dl309 in these cells (FIG. 5).

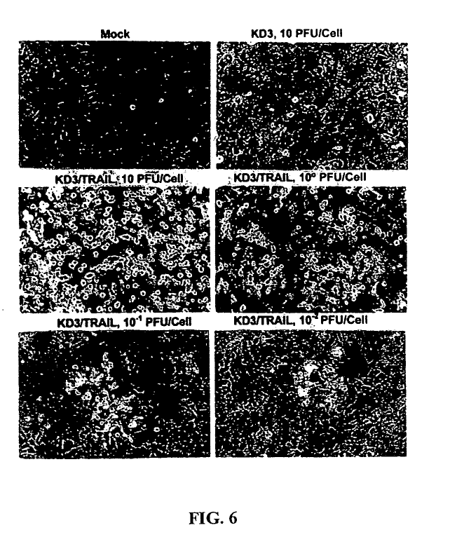

KD3/TRAIL kills the surrounding cells very efficiently but does not kill the infected cell at low MOI. Next, to dissect the overall cell killing effect of VRX-013, the efficacy of cell killing by VRX-013 or KD3 within the time-frame of one round of replication was compared. Hep3B cells were mock-infected or infected with dilutions of VRX-013 or KD3 ranging from 10 to 10⁻⁴ PFU/cell. The cytopathic effect (CPE) seen at 2 days p.i. is shown in FIG. 6. With KD3 at 10 PFU/cell, CPE was only beginning to become apparent, with a few cells rounding up (compare to mock infection). At 1 PFU/cell and lower MOIs there was no CPE in KD3-infected cells. With VRX-013, there was very extensive cell killing at 10 and 1 PFU/cell and even at 10−1 and 10−2 PFU/cell there were foci of rounded up cells (FIG. 6). Although not apparent in the figure, most of these cells had the “blebby” appearance of apoptotic cells. At 2 days p.i., Ads will not have had time to proceed through the process of infection, lysis, infection of surrounding cells, and CPE. Therefore, it is probable that the foci seen at 10⁻¹ and 10⁻² PFU/cell are the result of infection of a single cell with VRX-013, secretion of TRAIL, and TRAIL-induced apoptosis of surrounding cells. It is also possible that TRAIL expressed on the plasma membrane kills contiguous uninfected cells.

To determine whether or not VRX-013 kills the original infected cell, A549 cells were infected with VRX-013 or KD3 at 10⁻² PFU/cell and examined at 4 days p.i. Infected cells were fixed in methanol containing DAPI to visualize nuclei, then they were immunostained for the Adenovirus E2-coded DNA-binding protein (DBP). At 4 days p.i. there should be sufficient time for VRX-013 and KD3 to replicate in the originally-infected cell, lyse it, infect surrounding cells, and express DBP. As shown in FIG. 7 (top two panels) with KD3 most of the cells shown expressed DBP, indicating that the vector had spread from the original cell. With VRX-013, about eight nuclei that were stained positively for DBP are shown, five of them indicated by the arrows (FIG. 7, bottom two panels). In FIG. 7, VRX-013 is referred to as KD3/TRAIL. At this low MOI this frequency of infected cells is likely to arise from an originally infected cell.

To further address the question of whether or not VRX-013 kills the original infected cell, KB, HepG2 and SW1116 cells were infected with at 0.1, 10, 1.0 PFU/cell respectively, and at 2 days p.i. fixed and stained with DAPI to visualize nuclei and immunostained for the Adenovirus E2-coded DNA-binding protein (DBP). The DAPI panels show large intact nuclei that are immunostained for DBP, showing that these are infected cells. These infected cells are surrounded by many apoptotic nuclei with fragmented chromatin appearing as brightly stained objects that are out of the plane of focus (FIG. 8); these latter cells are not immunostained for DBP, and thus they were uninfected. Thus, the infected cells are not apoptotic, and the uninfected cells are apoptotic.

These results suggest three conclusions: (1) TRAIL does not induce apoptosis in the VRX-013-infected cell at low MOI; (2) VRX-013 can spread from cell-to-cell; and (3) TRAIL secreted from infected cells and/or expressed on the surface of infected cells induces apoptosis in surrounding cells.

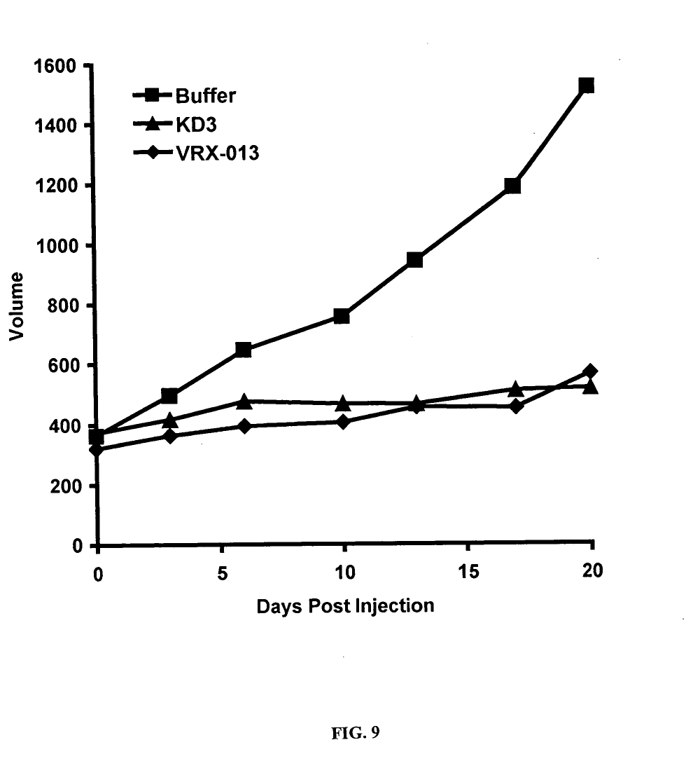

VRX-013 suppresses the growth of Hep3B xenografts in nude mice. To test the anti-tumor potential of VRX-013, subcutaneous Hep3B xenografts were established in nude mice. After 3 weeks, established tumors of average size of 300 mm³ were injected intratumorally with 5×10⁹ PFU/injection of KD3, VRX-013, or vehicle. Injections were repeated 5 times at 2 day intervals (total dose of 2.5×10¹⁰ PFU/tumor). At day 20 the mock-infected animals were terminated because the tumors became too large. As shown in FIG. 9, both KD3 and VRX-013 had significant tumor growth-retarding effect; tumors treated with either KD3 or VRX-013 grew 1.4-fold versus the about 4-fold growth of the controls.

It is also evident that KD3 and VRX-013 are equipotent in vivo. In evaluating this result, it was important to bear in mind that expression of ADP enhances the ability of replication-competent vectors to suppress the growth of tumors in nude mice (Doronin et al., 2000). However, as discussed earlier, VRX-013 makes somewhat less ADP than does KD3. Since VRX-007 was as effective as KD3 in suppressing the Hep3B tumors in nude mice (FIG. 9), it follows that VRX-013 has a feature that compensates for the reduced level of ADP. Most likely, this feature is synthesis of TRAIL. Therefore, the data in FIG. 9 indicate that TRAIL expressed by VRX-013 has a suppressive effect on tumors.

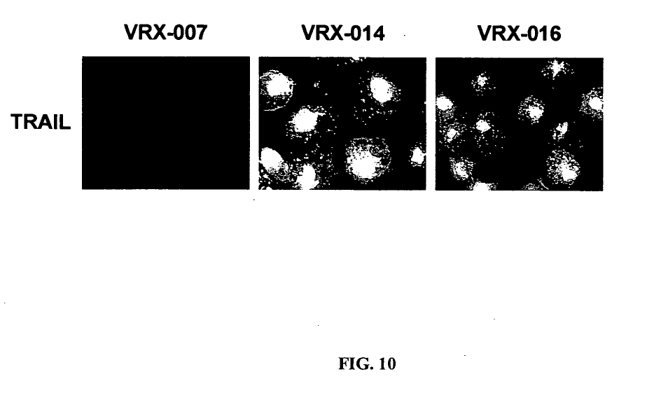

VRX-014 and VRX-016 express low levels of TRAIL, high levels of ADP, and they spread from cell-to-cell as efficiently as KD3 and VRX-007, respectively. As discussed, VRX-013 expresses large amounts of TRAIL but reduced levels of ADP. VRX-014 and VRX-016 were constructed to make high amounts of ADP but lower levels of TRAIL. As shown in the immunofluorescence experiment in FIG. 10, TRAIL was apparent in cells infected with VRX-014 or VRX-016. The TRAIL appeared on some membrane structures, possibly in the Golgi apparatus, and in putative vesicles. This immunostaining is specific to TRAIL, because it was not seen in VRX-007-infected cells (FIG. 10). The staining was not as bright as that seen with VRX-013-infected cells (see FIG. 2), suggesting that TRAIL is expressed in lesser amounts by VRX-014 and VRX-016 than by VRX-013.

These viruses were examined for their ability to synthesize ADP. As shown in the western blot in FIG. 11, VRX-014 synthesized at least as much and perhaps more ADP as did KD3. Similarly, VRX-016 synthesized as much ADP as did VRX-007. Since KD3 (Doronin et al., 2000) and VRX-007 express more ADP than wild-type Ad5 or the Ad5 mutant dl309 which expresses wild-type levels of ADP, it follows that VRX-014 and VRX-016 express more ADP than wild-type Ad5. VRX-013 synthesized less ADP than did KD3, VRX-014, or VRX-016.

It is noted that VRX-013 and VRX-014 have the same E1A mutation but that the orientation of the adp and trail gene in the E3 region is opposite. VRX-016 has the wild-type E1A gene, as does VRX-007, and VRX-016 has the same orientation of the adp and trail genes as does VRX-014.

The important design feature of these vectors is that the relative amount of expression of genes inserted into the E3 transcription unit can be predetermined by the orientation of the gene. Genes on the left will be expressed at higher levels than genes on the right (see FIG. 1A).

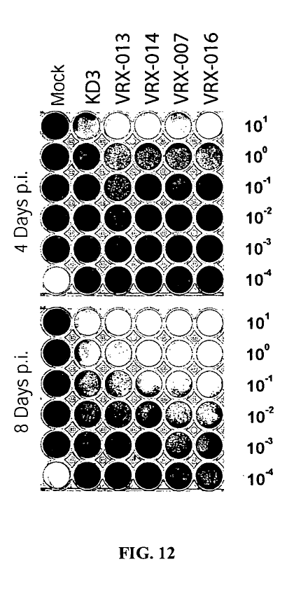

The relative levels of TRAIL and ADP synthesis has a significant effect on the timing at which these vectors lyse cells and spread from cell-to-cell. This effect is illustrated in the “vector spread” experiment shown in FIG. 12. Monolayers of human DLD-1 cancer cells were infected with different multiplicities of KD3, VRX-013, VRX-014, or VRX-016 ranging from 10 PFU/cell to 10⁻⁴ PFU/cell. Cells were stained with crystal violet at 4 days and 8 days p.i. At 4 days p.i., VRX-013 induced more cell lysis than did the other viruses; e.g., more of the monolayer had lifted off the plate with 10⁰ and 10⁻¹ PFU/cell of VRX-013 compared to the other viruses. This effect is very likely due to TRAIL-induced apoptosis. However, at 8 days, the other viruses induced more cell lysis (cytopathic effect, or cells detached from the dish) at low multiplicities of infection than did VRX-013 (FIG. 12). The ability of VRX-014, VRX-007, and VRX-016 to “catch up” to VRX-013 in inducing cytopathic effect is very likely due to their increased ability to spread from cell-to-cell due to their much higher levels of ADP synthesis as compared to VRX-0 13.

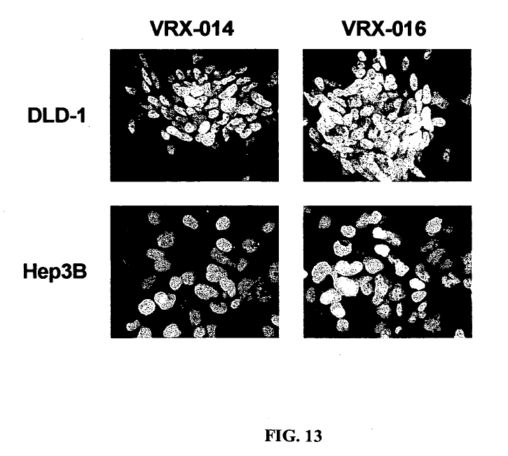

The ability of VRX-014 and VRX-016 to spread is further illustrated in the immunofluorescence experiment shown in FIG. 13. DLD-1 cells were infected with 2.2×10⁻⁴ PFU/cell of VRX-014 or 2.4×10⁻³ PFU/cell of VRX-016. Hep3B cells were infected with 6.6×10⁻⁴ PFU/cell of VRX-014 or 7.2×10⁻⁴ PFU/cell of VRX-016. At 3.5 days p.i., the cells were fixed and immunostained for the Adenovirus E1A protein. Expression of the E1A protein indicates that the cell has been infected. With both VRX-014 and VRX-016 and with both cell lines, foci of E1A-expressing infected cells were apparent (FIG. 13). Considering that the cells were infected with multiplicities of infection in the range of 10-3 to 104 PFU/cell, these foci no doubt arose from the initial infection of one or a few cells, vector replication in these cells, then vector spread to other cells. Thus, although VRX-014 and VRX-016 express TRAIL, the vectors are able to spread to surrounding cells and replicate in those cells.

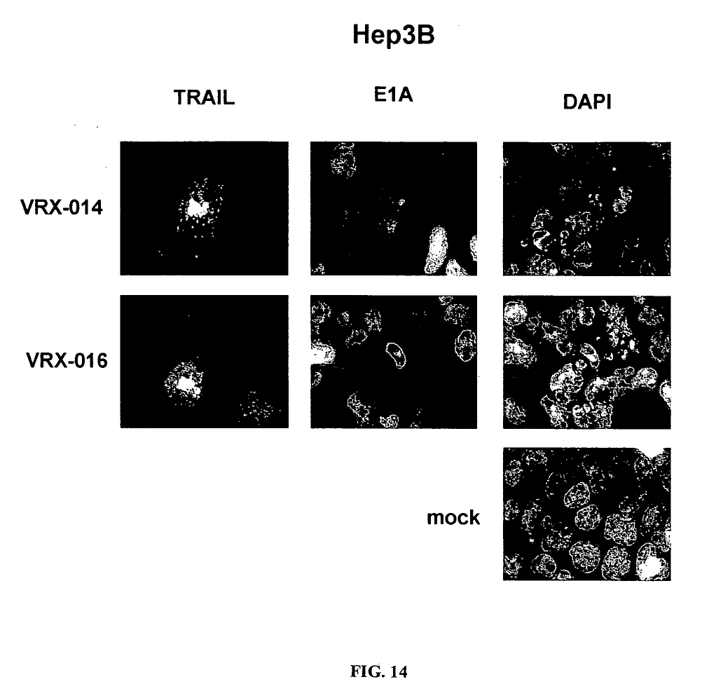

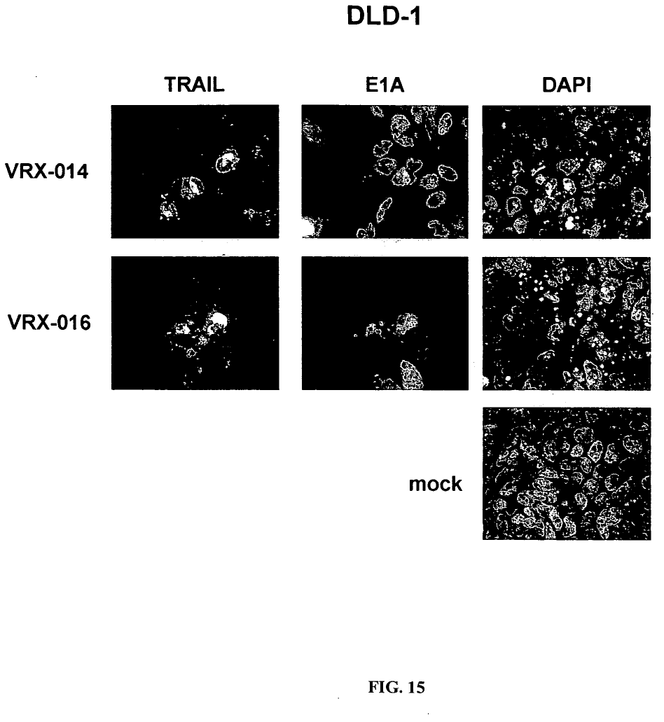

Having shown that VRX-014 and VRX-016 express TRAIL and spread from cell-to-cell, it was next addressed whether the TRAIL produced by these vectors is able to induce apoptosis in cells neighboring the infected cell. Hep3B or DLD-1 cells were mock-infected or infected with low multiplicities of infection of VRX-014 or VRX-016. At 3 days p.i., the cells were fixed, the nuclei were stained with DAPI, and the infected cells were double immunostained using a rabbit antiserum specific to TRAIL and a mouse monoclonal antiserum specific to E1A. For the infections, the same field of cells is shown for TRAIL, E1A, and DAPI (FIGS. 14 and 15).

With VRX-014 in Hep3B cells, there were one or two cells that were stained for TRAIL (FIG. 14, top left panel) and E1A (top middle panel), and these cells had non-apoptotic nuclei (top right panel). There were other cells that were positive for E1A (top middle panel) and had non-apototic nuclei (top right panel). On the other hand, there were about five apoptotic nuclei (top right panel) from cells that were not immunostained for E1A. For VRX-016, there were TRAIL-positive and E1A-positive cells that had non-apoptotic nuclei, and several cells that were not stained for TRAIL or E1A that had apoptotic nuclei (FIG. 14, middle left, middle, and right panels). Mock-infected cells had non-apoptotic nuclei, as expected. This same pattern was observed with the DLD-1 cells. With both VRX-014 (FIG. 15, top three panels) and VRX-016 (FIG. 15, middle three panels), there were TRAIL- and E1A-positive cells with non-apoptotic nuclei and there were TRAIL- and E1A-negative cells that had apoptotic nuclei.

These results are similar to those obtained with VRX-013. That is, (i) infected cells express TRAIL, (ii) infected cells as indicated by expression of an adenovirus-coded protein (E1A in FIGS. 14 and 15) in general do not have apoptotic nuclei, possibly because they are protected from TRAIL-induced apoptosis by the Adenovirus E1B-19K anti-apoptotic protein, and (iii) uninfected cells neighboring the infected cells have apoptotic nuclei, presumably induced either by release of TRAIL from infected cells or because of direct contact with TRAIL expressed on the surface of infected cells.

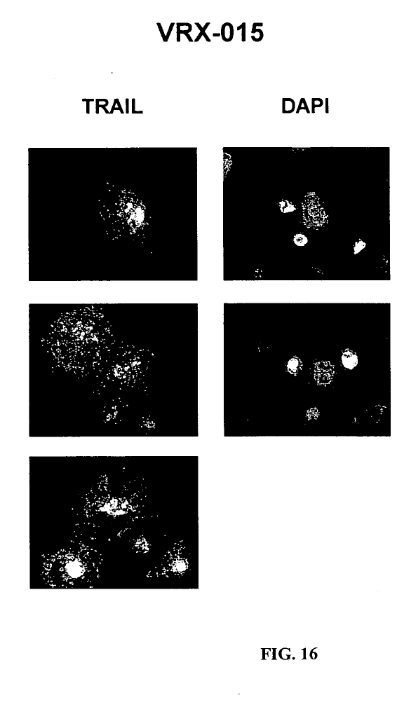

VRX-015 expresses TRAIL which induces apoptosis in cells neighboring the TRAIL-expressing cells. As discussed earlier, the inventors have isolated many plaques that are expected to be VRX-015. The virus from one of the plaques expresses TRAIL as indicated by immunofluorescence (FIG. 16, three left panels). The TRAIL is localized in the Golgi apparatus, in vesicles, and in some cells on the plasma membrane. Of interest, the nuclei of the TRAIL expressing cells appeared to be non-apoptotic, whereas some nuclei in surrounding cells were apoptotic (FIG. 16, compare the top two panels; also compare the middle two panels). Thus, it is very likely that the inventors have obtained VRX-015 and that it will exhibit the vector replication and TRAIL-induced apoptosis properties of the other TRAIL-expressing vectors. It is expected that VRX-015 will express as much TRAIL as does VRX-013, and because it has wild-type E1A rather than mutant E1A, that VRX-015 will replicate more efficiently in a larger number of different cell types than will VRX-013.

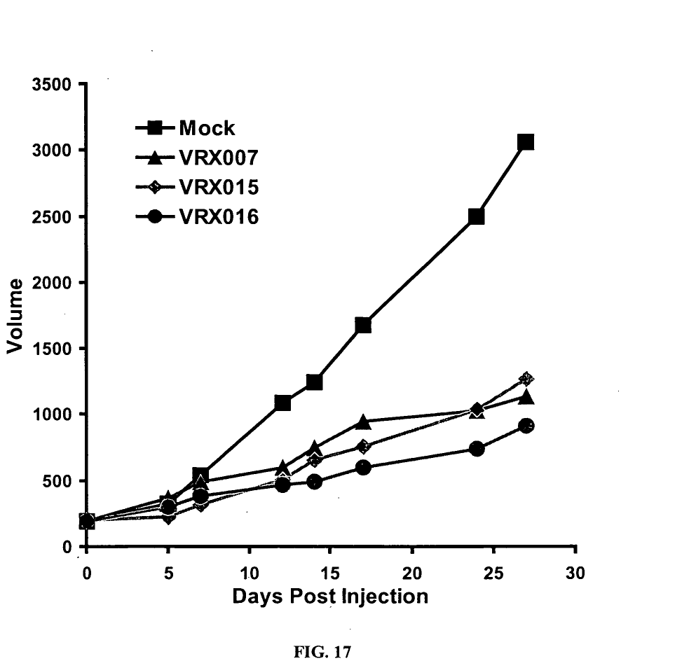

Tumor suppression in nude mice. The TRAIL-expressing vectors, VRX-015 and VRX-016, with VRX-007, were examined for their ability to suppress tumors in nude mice (FIG. 17). It was expected that the TRAIL vectors would be superior to VRX-007, especially VRX-016; however, the TRAIL vectors were statistically indistinguishable from VRX-007; all three vectors were statistically different from the mock-injected tumors. With VRX-015, the TRAIL gene is on the left of the ADP gene in the E3 region. VRX-015 is in a KD3 background (i.e. two small deletions in the E1A gene). With VRX-016, the ADP gene is on the left and the TRAIL gene is on the right. VRX-016 is in a VRX-007 background (wild type E1A).

To improve on the effectiveness of these vectors in vivo alternative approaches are employed. Such approaches involve using the amino acid sequence coding for the entire region of TRAIL rather than wild-type TRAIL gene in the VRX-016 vector.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference.

U.S. Pat. No. 4,601,978

U.S. Pat. No. 5,013,652

U.S. Pat. No. 5,089,397

U.S. Pat. No. 5,354,855

U.S. Pat. No. 5,359,046

U.S. Pat. No. 5,464,758

U.S. Pat. No. 5,464,758

U.S. Pat. No. 5,512,483

U.S. Pat. No. 5,521,284

U.S. Pat. No. 5,559,027

U.S. Pat. No. 5,559,904

U.S. Pat. No. 5,795,715

U.S. Pat. No. 5,851,796

U.S. Pat. No. 5,877,018

U.S. Pat. No. 5,925,565

U.S. Pat. No. 5,925,565

U.S. Pat. No. 5,935,819

U.S. Pat. No. 5,935,819

U.S. Pat. No. 5,971,122

U.S. Pat. No. 6,013,516

U.S. Pat. No. 6,133,027

U.S. Pat. No. 6,207,146

U.S. Pat. No. 6,440,741

U.S. Pat. No. 6,649,260

U.S. patent application Ser. No. 09/351,778

U.S. patent application Ser. No. 09/351,778

Alemany et al., Nat. Biotechnology, 18:723-727, 2000.

Andriani et al., JNCI, 93(17):1314-24, 2001.

Arai et al., Proc. Nat'l Acad. Sci. USA, 94:13862-13867, 1997.

Armeanu et al., Cancer Res., 63:2369-2372, 2001.

Ashkenazi et al., J. Clin. Invest., 104:155-162, 1999.

Bakhshi et al., Cell, 41(3):899-906, 1985.

Bedzyk et al., J. Biol. Chem., 265(30):18615-18620, 1990.

Bischoff et al., Science, 274:373-376, 1996.

Bodmer et al., J. Biol. Chem., 275:20632-206307, 2000.

Bodnar et al., Science, 279:349-352, 1998.

Braselmann et al., Proc. Nat'l Acad. Sci. USA, 90(5):1657-1661, 1993.

Burbage et al., Leuk. Res., 21(7):681-690, 1997.

Caldas et al., Nat. Genet., 8(1):27-32, 1994.

Caplen et al., Gene, 252(1-2):95-105, 2000.

Chaudhary et al., Proc. Nat'l Acad. Sci. USA, 87(3):1066-1070, 1990.

Cheng et al., Cancer Res., 54(21):5547-5551, 1994.

Cleary and Sklar, Proc. Nat. Acad. Sci. USA, (21):7439-7443, 1985.

Cleary et al., J. Exp. Med., 164(1):315-320, 1986.

Cong et al., Hum. Mol. Genet., 8:137-142, 1999.

Corces et al., Proc. Nat. Acad. Sci. USA, 78:7038-7042, 1981.

Counter et al., Oncogene, 16:1217-1222, 1998.

Djeha et al., Mol. Ther., 3:233-240, 2001.

Doronin et al., J. Virol., 74:6147-6155, 2000.

Doronin et al., J. Virol., 75(7):3314-3324, 2001.

Elbashir et al., Nature, 411(6836):494-498, 2001.

Fire et al., Nature, 391:806-811, 1998.

Forster and Symons, Cell, 49(2):211-220, 1987.

Freytag et al., Human Gene Ther., 9:1323-1333, 1998.

Gerlach et al., Nature (London), 328:802-805, 1987.

Goldberg et al., Cancer Res., 61:1334-1337, 2001.

Goodrum and Ornelles, J. Virol., 72:9479-9490, 1998.

Greenberg et al., Mol Endo, 8:230-9, 1994.

Greenberg et al., Proc Nat'l Acad. Sci. USA, 92:3439-43, 1995.

Griffith and Broghammer, Mol. Ther., 4:257-266, 2001.

Griffith et al., J. Immunol., 165(5):2886-2894, 2000.

Griffith et al., J. Immunol., 165:2886-2894, 2000.

Grishok et al., Science, 287:2494-2497, 2000.

Hahn et al., Nature, 400:464-468, 1999.

Harada and Berk, J. Virol., 73:5333-5344, 1999.

Harlow, J. Virol., 55:533-546, 1985.

Harrington et al., Gene Dev., 11:3109-3115, 1997.

Hauck and Stanners, J. Biol. Chem., 270:3602-3610, 1995.

Haviv et al., Cancer Res., 61:8361-8365, 2001.

Hawkin and Hermiston Gene, 81:142-148, 2001.

Hinoda et al., Japanese J. Clin. Oncol., 21:75-81, 1991.

Holmgren et al., Proc. Nat. Acad. Sci. USA, 78:3775-3778, 1981.

Horikawa et al., Cancer Res., 59:826-830, 1999.

Horwitz, In: Fields Virology, Field et al. (Eds.), Lippencott-Raven, Philadelphia, 2149-2171, 1996.

Howe et al., Proc. Nat'l Acad. Sci. USA, 87:5883-5887, 1990.

Hussussian et al., Nat. Genet., 8(1):15-21, 1994.

Jessup and Thomas, Cancer and Metastasis Rev,. 8:263-280, 1989.

Jones and Shenk, Cell, 17:683-689, 1979.

Joyce, Nature, 338:217-244, 1989.

Kagawa et al., Cancer Res., 61:3330-3338

Kamb et al., Nat. Genet., 8(1):23-2, 1994.

Kerr et al., Br. J. Cancer, 26(4):239-257, 1972.

Ketting et al., Cell, 99:133-141, 1999.

Khuri et al., Nat. Med., 6:879-885, 2000.

Kijima et al., Cancer Res., 59:4906-4911, 1999.

Kilian et al., Hum. Mol. Genet., 12:2011-2019, 1997.

Kim and Chech, Proc. Nat'l Acad. Sci. USA, 84(24):8788-8792, 1987.

Kim et al., Nat. Med., 4:1341-1342, 1998.

Krajcsi et al., J. Virol., 70:4904-4913, 1996.

Latham et al., Cancer Res., 60(2):334-41, 2000.

Lidor et al., Am. J. Obstet. Gynecol, 177(3):579-585, 1997.

Lin and Avery, Nature, 402:128-129, 1999.

Lis et al., Cell, 35:403-410, 1983.

Macejak and Sarnow, Nature, 353:90-94, 1991.

Mariani and Krammer, Eur. J. Immunol., 28:973-982, 1998.

Martin and Berk, Mol. Cell Biol., 19:3403-3414, 1999.

Massuda et al., Proc. Nat'l Acad. Sci. USA, 94(26):14701-14706, 1997.

McAlinden et al., J. Biol. Chem., 277(43):41274-41281, 2002.

Meyerson et al., Cell, 90:785-795, 1997.

Mirault et al., EMBO J., 1: 1279-1285, 1982.

Montgomery et al., Proc. Nat'l Acad. Sci. USA, 95:155-2-15507, 1998.

Morris et al., J. Biol. Chem., 274(1):418-423, 1999.

Nakamura et al., Science, 277:955-959, 1997.

Nakayama et al., Nature Genet., 18:65-68, 1998.

Neumaier et al., J. Biol. Chem., 263:3202-3207, 1988.

Nobori et al., Nature, 368(6473):753-756, 1994.

O'Shea et al., Science, 243(4890):538-542, 1989.

Oikawa et al., Biochem. Biophys. Res. Comm., 146:464-469, 1987.

Okamoto et al., Proc. Nat'l Acad. Sci. USA, 1(23):11045-1104994.

Orlow et al., Int. J. Oncol., 15(1):17-24, 1999.

Osaki et al., Cancer Res., 54:5258-5261, 1994.

Palaniyar et al., Comp. Biochem. Physiol. Mol. Integr. Physiol, 129(1):109-127, 2001.

Paxton et al., Proc. Nat'Acad. Sci. USA, 84:920-924, 1987.

PCT/US00/18971

Pelham and Bienz, EMBO J., 1:1473-1477, 1982.

Pelham, Cell, 30:517-528, 1982.

Pelletier and Sonenberg, Nature, 357:173-176, 1992.

Peteranderl and Nelson, Biochemistry, 31(48):12272-12276, 1992.

Remington's Pharmaceutical Sciences, 18th Edition, Chapter 61, pages 1289-1329, 1990.

Rennie et al., Mol Endo, 7:23-36, 1993.

Richards et al., Hum. Gene Ther., 6:881-893, 1995.

Robbins et al., Int. J. Cancer, 53:892-897, 1993.

Rogulski et al., Cancer Res., 60:1193-1196, 2000.

Sadowski et al., Nature, 335(6190):563-564, 1988.

Sarver et al., Science, 247:1222-1225, 1990.

Scanlon et al., Proc. Nat'l Acad. Sci. USA, 88:10591-10595, 1991.

Scaria et al., Virology, 191:743-753, 1992.

Schrewe et al., Mol. Cell. Biol., 10:2738-2748, 1990.

Schuur et al., J. Biol. Chem., 271(12):7043-7051, 1996.

Serrano et al., Nature, 366:704-707, 1993.

Serrano et al., Science, 267(5195):249-252, 1995.

Sharp and Zamore, Science, 287:2431-2433, 2000.

Sharp, Genes Dev., 13:139-141, 1999.

Shenk, In: Fields Virology, Field et al. (Eds.), Lippencott-Raven, Philadelphia, 2111-2148, 1996.

Sheridan et al., Science, 277:81 8-821, 1997.

Shinoura et al., Cancer Res., 59:3411-3416, 1999.

Shu et al., Biochemistry, 38(17):5378-5385, 1999.

Smith et al., Cancer, 9:121 1-1218, 1956.

Stewart et al., “Adenovirus structure by x-ray crystallography and electron microscopy.” in: The Molecular Repertoire of Adenoviruses, Doerfler et al., (ed)., Springer-Verlag, Heidelberg, Germany, p. 25-38.

Tabara et al., Cell, 99:123-132, 1999.

Takakura et al., Cancer Res., 59:551-557, 1999.

Thompson et al., J. Clin. Lab. Anal., 5:344-366, 1991.

Tollefson et al., In: Adenovirus methods and protocols, Wold (ed.), Humana Press, Inc., Totowa, N.J., 1998.

Tollefson et al., J. Virol. 70:2296-2306, 1996b.

Tollefson et al., J. Virol., 66:3633-3642, 1992.

Tollefson et al., J. Virol., 66:3633-3642, 1996a.

Tollefson et al., J. Virol., 220:152-162, 1996c.

Toth et al., Virol., 301(1):99-108, 2002.

Tsujimoto and Croce, Proc. Nat'l Acad. Sci. USA, 83(14):5214-5218, 1986.

Tsujimoto et al., Science, 228(4706):1440-1443, 1985.

Walczak et al., Nat. Med., 5:157-163, 1999.

Walczak et al., Nature Med., 3(2):157-163, 1999.

Webb and Smith, Lancet, 1: 1206-8, 1970.

Weinberg, Science, 254(5035):1138-1146, 1991.

Weinrich et al., Nature Genet., 17:498-502, 1997.

White, Semin. Virol. 8505-513, 1998.

Wilder and Norris, Cancer Res., 60:4167-4174, 2000.

Wildner, Ann. Med., 31:421-429, 1999.

Wiley et al., Immunity, 3(6):673-682, 1995.

WO 00/44914

WO 01/36646

WO 01/68836

WO 99/32619

Wold et al., In: Virus Replication: Frontiers in Molecular Biology, Cann (Ed.), Oxford University Press, Oxford, 200-232.

Wold et al., Trends Microbiol., 2:437-443, 1994.

Zhang and Chen, J. Biol. Chem., 274(32):22409-22413, 1999.

What is claimed is:

1. A replication-competent adenovirus vector comprising a tumor necrosis factor-related apoptosis-inducing ligand (TRAIL) coding region and an ADP coding region.

2. The vector of claim 1, wherein said TRAIL coding region and said ADP coding region are positioned under the control of adenovirus major late promoter (MLP).

3. The vector of claim 1, wherein said TRAIL coding region and said ADP coding region are positioned in the E3 region of said vector.

4. The vector of claim 3, wherein said TRAIL coding region is positioned upstream of said ADP coding region.

5. The vector of claim 3, wherein said TRAIL coding region is positioned downstream of said ADP coding region.

6. The vector of claim 1, wherein said TRAIL coding region is positioned under the control of adenovirus major late promoter (MLP), and said ADP coding region is positioned under the control of another promoter.

7. The vector of claim 1, wherein said ADP coding region is positioned under the control of adenovirus major late promoter (MLP), and said TRAIL coding region is positioned under the control of another promoter.

8. The vector of claim 1, wherein said vector lacks one or more of coding regions for the 6.7K, gp19K, RIDα, RIDβ or 14.7K proteins.

9. The vector of claim 8, wherein said vector lacks all of the coding regions for the 6.7K, gp19K, RIDα, RIDβ or 14.7K proteins.

10. The vector of claim 1, wherein said vector further comprises at least a first mutation in the E1A region, said mutation impairing binding of E1A to p300 and/or pRB.

11. The vector of claim 10, wherein said vector lacks coding regions for 6.7K, gp19K, RIDα, RIDβ or 14.7K proteins, and said TRAIL coding region is positioned upstream of said ADP coding region.

12. The vector of claim 1, wherein said vector lacks coding regions for 6.7K, gp19K, RIDα, RIDβ or 14.7K proteins, contains a wild-type E1A coding region, and said TRAIL coding region is positioned upstream of said ADP coding region.

13. The vector of claim 10, wherein said vector lacks coding regions for 6.7K, gp19K, RIDα, RIDβ or 14.7K proteins, and said TRAIL coding region is positioned downstream of said ADP coding region.

14. The vector of claim 1, wherein said vector lacks coding regions for 6.7K, gp19K, RIDα, RIDβ or 14.7K proteins, contains a wild-type E1A coding region, and said TRAIL coding region is positioned downstream of said ADP coding region.

15. The vector of claim 1, wherein said vector is oncolytic.

16. An adenoviral virion comprising a replication-competent adenoviral vector according to claim 1.

17. A host cell comprising the replication-competent adenoviral vector of claim 1

18. A method of inhibiting a hyperproliferative cell comprising contacting said cell with a second cell infected with a replication-competent adenovirus vector comprising a tumor necrosis factor-related apoptosis-inducing ligand (TRAIL) coding region and an ADP coding region.

19. The method of claim 18, wherein inhibiting comprises inhibiting cell division, inhibiting cell growth, inducing cell cycle arrest, inducing apoptosis or lysing.

20. The method of claim 18, wherein said hyperproliferative cell is a cancer cell.

21. The method of claim 18, wherein said second cell is a cancer cell.

22. The method of claim 20, wherein said cancer cell is a lung cancer cell, a prostate cancer cell, a colon cancer cell, an ovarian cancer cell, a testicular cancer cell, a brain cancer cell, a stomach cancer cell, a uterine cancer cell, a breast cancer cell, an esophageal cancer cell, a head & neck cancer cell, a pancreatic cancer cell, a liver cancer cell, a kidney cancer cell, a skin cancer cell or a blood cancer cell.

23. A method of treating a subject with a hyperproliferative cell disorder comprising administering to said subject a replication-competent adenovirus vector comprising a tumor necrosis factor-related apoptosis-inducing ligand (TRAIL) coding region and an ADP coding region.

24. The method of claim 23, wherein said hyperproliferative all disorder is cancer.

25. The method of claim 24, wherein said cancer is lung cancer, prostate cancer, colon cancer, ovarian cancer, testicular cancer, brain cancer, stomach cancer, uterine cancer, breast cancer, esophageal cancer, head & neck cancer, pancreatic cancer, liver cancer, kidney cancer, skin cancer or blood cancer.

26. The method of claim 23, wherein said subject is a human.

27. The method of claim 23, further comprising administering to said subject a second therapy.

28. The method of claim 23, wherein said second therapy is chemotherapy, radiotherapy, immunotherapy, hormonal therapy, gene therapy or surgery.

29. The method of claim 23, wherein said second therapy is provided prior to said replication-competent adenovirus vector.

30. The method of claim 23, wherein said second therapy is provided after said replication-competent adenovirus vector.

31. The method of claim 23, wherein said second therapy is provided at the same time as said replication-competent adenovirus vector.

32. The method of claim 23, wherein said replication-competent adenovirus vector is administered more than once.

33. The method of claim 23, wherein said replication-competent adenovirus vector is administered intratumorally, locally to said tumor, regionally to said tumor or systemically.

34. The method of claim 23, wherein said replication-competent adenovirus vector is administered intravenously, intraarterially, intramuscularly, intralymphatically, intraperitoneally or subcutaneously.

35. The method of claim 23, wherein said TRAIL coding region and said ADP coding region are positioned under the control of adenovirus major late promoter (MLP).

36. The method of claim 23, wherein said TRAIL coding region and said ADP coding region are inserted into the E3 region.

37. The method of claim 36, wherein said TRAIL coding region is positioned upstream of said ADP coding region.

38. The method of claim 36, wherein said TRAIL coding region is positioned downstream of said ADP coding region.

39. The vector of claim 23, wherein said TRAIL coding region is positioned under the control of adenovirus major late promoter (MLP), and said ADP coding region is positioned under the control of another promoter.

40. The vector of claim 23, wherein said ADP coding region is positioned under the control of adenovirus major late promoter (MLP), and said TRAIL coding region is positioned under the control of another promoter.

41. The method of claim 23, wherein said vector lacks one or more of coding regions for the 6.7K, gp19K, RIDα, RIDβ or 14.7K proteins.

42. The method of claim 41, wherein said vector lacks all of the coding regions for the 6.7K, gp19K, RIDα, RIDβ or 14.7K proteins.

43. The method of claim 23, wherein said vector further comprises at least a first mutation in the E1A region, said mutation impairing binding of E1A to p300 and/or pRB.

44. The method of claim 24, wherein said cancer is a multi-drug resistant cancer.

45. The method of claim 23, wherein treating comprises reducing tumor size, reducing tumor growth, inducing remission, inducing tumor necrosis, or prolonging patient survival.

46. A method of rendering an inoperable tumor operable comprising administering to a subject a replication-competent adenovirus vector comprising a tumor necrosis factor-related apoptosis-inducing ligand (TRAIL) coding region and an ADP coding region.

47. A method of treating metastatic cancer in a subject comprising administering to subject a replication-competent adenovirus vector comprising a tumor necrosis factor-related apoptosis-inducing ligand (TRAIL) coding region and an ADP coding region.

48. A method of preventing cancer in a subject at risk thereof comprising administering to said subject a replication-competent adenovirus vector comprising a tumor necrosis factor-related apoptosis-inducing ligand (TRAIL) coding region and an ADP coding region.

49. A method of treating recurrent cancer in a subject comprising administering to said subject a replication-competent adenovirus vector comprising a tumor necrosis factor-related apoptosis-inducing ligand (TRAIL) coding region and an ADP coding region.
